(19) United States
(12) Patent Application Publication
(10) Pub. No.: US 2018/0072675 A1
(43) Pub. Date: Mar. 15, 2018

(54) ANTIMICROBIAL PYRIDINOHYDRAZIDE AND HYDRAZOMETHYLPYRIDINE-BASED AGENTS

(71) Applicants: Board of Supervisors of Louisiana State University and Agricultural and Mechanical College, Baton Rouge, LA (US); THE UNIVERSITY OF NEW ORLEANS, NEW ORLEANS, LA (US)

(72) Inventors: DONNA M. NEUMANN, NEW ORLEANS, LA (US); BRANKO S. JURSIC, NEW ORLEANS, LA (US)

(21) Appl. No.: 15/561,654

(22) PCT Filed: Mar. 25, 2016
(86) PCT No.: PCT/US16/24175

§ 371 Date: Sep. 26, 2017

Related U.S. Application Data
(60) Provisional application No. 62/139,179, filed on Mar. 27, 2015.

(51) Int. Cl. C07D 213/81; C07D 213/72; C07D 213/53; C07D 213/89

(52) U.S. Cl. CPC C07D 213/81; C07D 213/89; C07D 213/53; C07D 213/72

(57) ABSTRACT

A class of modified salicylaldehyde derivatives has also been synthesized and a series of modified pyridine-based hydrazones identified that have potent antimicrobial activity against multiple Candida spp. These compounds have been characterized using fungal growth inhibition assays, mammalian cell toxicity assays, time-kill assays and synergy studies of these novel pyridine-based hydrazones on both azole-susceptible and azole-resistant fungal species. Effectiveness of these compounds in inhibiting the growth of protozoal parasites was also found.

### CROSS-REFERENCE TO RELATED APPLICATIONS

This application claims priority to U.S. Provisional Patent Application Ser. No. 62/139,179 entitled “ANTIMICROBIAL PYRIDINOHYDRAZIDE AND HYDRAZOMETHYLPYRIDINE-BASED AGENTS” filed on Mar. 27, 2015, the entirety of which is hereby incorporated by reference.

### TECHNICAL BACKGROUND

The present disclosure is generally related to pyridinohydrazide and hydrazomethylpyridine-based antimicrobial compounds. The present disclosure is further related to protected pyridine hydrazidones of 5-substituted salicylaldehyde antimicrobial compounds. The disclosure is further related to pharmaceutical compositions comprising the pyridinohydrazide and hydrazomethylpyridine-based antimicrobial compounds.

### BACKGROUND

The incidence rate for fungal infections has increased dramatically in the last few decades. For instance, recent analyses of hospital data in France revealed that the annual rate of invasive fungal infections increased by 16% and the annual rate of deaths from invasive fungal infections increased 33% in the last decade (Biter et al., Emerg. Infect. Dis. (2014) 20: 1149-1155), and it is reasonable to assume that world trends are similar. The most common culprits for the onset of invasive infections arise from the Candida spp. Candidiasis is most commonly associated with Candida albicans, Candida glabrata and Candida krusei microorganisms (Comely et al., Mycoses 2014 57: 79-89; Comely et al., J Antimicrob. Chemother. (2007) 60: 363-369).

The immune-compromised, specifically transplant recipients, cancer patients and those infected with AIDS, are particularly susceptible to candidiasis infections (Chakrabarti et al., Nippon Ishinkin. Gakkai Zasshi (2008) 49: 165-172; Chandesris et al., Rev. Prat. (2007) 57: 1653-1664). While classes of antifungal therapies, such as the polyenes, azoles and the echinocandins, are available for the treatment of invasive Candida spp. infections, many have adverse effects or other drawbacks associated with their use, including nephrotoxicity, hepatotoxicity and the development of resistance in the target microorganism (Roemer & Krysan Cold Spring Harb. Perspect. Med. (2014) 4; Cha & Sobel Expert Rev. Anti. Infect. Ther. (2004) 2: 357-366). In addition, oral candidiasis is one of the most frequent opportunistic infections associated with HIV and AIDS, and these infections have largely been controlled with the use of topical polyenes or systemic azoles (White T C. Antimicrob. Agents Chemother. (1997) 41: 1482-1487). However, the overuse of azoles in the treatment of oral candidiasis has led to the development of drug resistance. For example, it has been reported that up to 33% of AIDS patients have azole-resistant oral candidiasis infections (White T C. Antimicrob. Agents Chemother. (1997) 41: 1482-1487). This resistance to the azoles stems not only from the drugs' fungistatic effects, but also from overexpression of efflux pumps (Eddouzi et al., Antimicrob. Agents Chemother. (2013) 57: 3182-3193) and point mutations in the ERG11 gene (Xiang et al., FEMS Yeast Res. (2013) 13: 386-393) in Candida albicans. In addition, Candida glabrata develops resistance during prolonged treatment with azole antifungals (Bennett et al., Antimicrob. Agents Chemother. (2004) 48: 1773-1777). For these reasons, there is a continuous demand for the discovery of novel therapeutics to treat Candida spp. infections, particularly if they have a different method of action than the azoles.

Some literature data suggest that simple Schiff bases possess antimicrobial activity and might have different biological targets than currently available antifungal agents (Verma et al., J Pharm Bioallied. Sci (2014) 6: 69-80). Others have previously studied the antimicrobial activity of a limited number of salicylaldehyde hydrazones, although the mechanisms of action for these derivatives have not been clearly defined (Pelttari et al., Z. Naturforsch. C. (2007) 62: 487-497). It was suggested that the activity of these molecules may be based on the formation of Schiff bases with important amino groups of the microbial cells.

To further explore this structure-activity relationship and to identify possible pharmacophores, a large series of salicylaldehyde (Backes et al., Bioorg. Med. Chem. (2014); 22: 4629-4636) and formylpyridinetrione hydrazones (Neumann et al., Bioorg. Med. Chem (2014); 22: 813-826) were synthesized and then analyzed for their ability to inhibit fungal growth of both azole-susceptible and azole-resistant species of Candida. Additionally, potential mechanisms of action for these compounds were explored. Many of these analogs showed excellent growth inhibition with low mammalian cell toxicity. However, this activity did not extend to azole-resistant species of Candida. Furthermore, mechanistic studies indicated that these compounds were likely fungistatic, as with azoles. Therefore, a new structure-activity relationship study was designed using modified salicylaldehyde derivatives not previously studied, with the goal of determining the pharmacophore required to produce antifungal activity against azole-resistant fungi. A series of modified pyridine-based hydrazones were identified that had potent fungicidal antifungal activity against multiple Candida spp.

### SUMMARY

Schiff base derivatives have been shown to possess antimicrobial activity, and these derivatives include a limited number of salicylaldehyde hydrazones. A large series of salicylaldehyde and formylpyridinetrione hydrazones had been synthesized and analyzed for their ability to inhibit fungal growth of both azole-susceptible and azole-resistant species of Candida. While many of these analogs showed excellent growth inhibition with low mammalian cell toxicity, their activity did not extend to azole-resistant species of Candida. A novel class of modified salicylaldehyde derivatives has now been synthesized and a series of modified pyridine-based hydrazones identified that have potent antimicrobial activity against multiple Candida spp. These compounds have been characterized using fungal growth inhibition assays, mammalian cell toxicity assays, time-kill assays and synergy studies of these novel pyridine-based hydrazones on both azole-susceptible and azole-resistant fungal species.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIG. 1 illustrates scheme 1 for the preparation of hydrazides from corresponding carboxylic acids.

FIG. 2 illustrates scheme 2 for the preparation of sulfonohydrazides.

FIG. 3A illustrates scheme 3 for the preparation method for 2-hydroxybenzylidenepyridinohydrazides.

FIG. 3B illustrates modification of a Hickman still.

FIG. 4 illustrates scheme 4 for the preparation of members of a hydrazonomethylpyridine library.

FIG. 5 schematically illustrates the preparation of protected salicylaldehydes

FIG. 6 schematically illustrates a method for the preparation of the hydrazidone prodrugs 1-4.

FIG. 7 schematically illustrates a method for the preparation hydrazidone prodrugs 1-4.

FIG. 8, Panels A-G, illustrates the evaluation of mammalian cell toxicity of select active compounds. Cytotoxicity was determined using non-cancerous Vero cells (kidney-african green monkey) and liver cells (Hep-2G) in accordance with Promega CellTiter 96 Non-Radioactive Cell Proliferation Assay (cat # G4000). Compounds were diluted in media and all assays were done in triplicate. Average values are presented, with ±SD. Cells were incubated in the presence of the compounds for 24 h at 37° C. and 5% CO₂. Tetrazolium dye solution was added to each well and allowed to incubate for 1-4 h. Solubilization/Stop solution was added and allowed to sit at room temperature for 1 h. Formazan product was scored spectrophotometrically with an automatic plate reader set at 570 nm. Positive control wells for cell death were briefly treated with 0.1% saponin, immediately prior to the addition of dye solution. Wells containing 1% DMSO and no drug were used as a negative control and the average absorbance at 570 nm were set to 100%. The percent of cells viable were calculated as a ratio between the average absorption at 570 nm of the drug containing wells/average absorption at 570 nm of the control (no drug). Ratios were multiplied by 100 to give the values as a percentage as expressed on the y-axis.

FIG. 9 illustrates time-kill assays, for representative analogs that were active against the azole-resistant TW17, to determine the cidal or static nature of the analog tested. 100 μl of each sample was spread onto YM agar plates with a sterile bent spreading rod. Plates were incubated at 35° C. overnight and colonies were then counted on each plate.

FIGS. 10-23 illustrate the results of the compounds of the disclosure tested for toxicity to mammalian cells.

FIG. 24 illustrates the chemical structures of some compounds used in the experiments of the disclosure.

FIG. 25 illustrates the efficacy of certain compounds (25 μM each) of the disclosure in reducing the proliferation of T cruzi.

FIG. 26 illustrates the efficacy of certain compounds (25 μM each) of the disclosure in reducing the proliferation of Tcruzi.

FIG. 27 illustrates chemical structures of the compounds SA81, SA82, SA92 (alternative designation AR4), SA105 (alternative designation AR5), and SA113 that reduce protozoal proliferation.

FIG. 28 is a graph illustrating the growth kinetics of L. amazonensis under 9 different treatments over an 8 day period. Density reported in parasites/mL×10⁵.

FIG. 29 is a graph illustrating parasite density on Day 7 of growth under each treatment option. All compounds were at 50 μM concentration unless otherwise stated. Density reported in parasites/mL×10⁵.

FIG. 30 is a graph illustrating the effects of combining compounds of the disclosure based on structural compatibilities. Density reported in parasites/mL×10⁵.

FIG. 31 is a graph illustrating compounds that showed positive effects retested at 25 μM to test for potency. Density reported in parasites/mL×10⁵.

FIG. 32 illustrates compounds of the disclosure.

FIG. 33 illustrates compounds of the disclosure.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law, allowing for the presence of more than that which is recited so long as basic or novel characteristics of that which is recited is not changed by the presence of more than that which is recited, but excludes prior art embodiments.

### Definitions

The term “substantially” as used herein means completely or almost completely; for example, a composition that is “substantially free” of a component either has none of the component or contains such a trace amount that any relevant functional property of the composition is unaffected by the presence of the trace amount, or a compound is “substantially pure” if there are only negligible traces of impurities present.

The terms “treating” or “treatment” within the meaning herein refers to an alleviation of symptoms associated with a disorder or disease, or inhibition of further progression or worsening of those symptoms, or prevention or prophylaxis of the disease, infection, or disorder, or curing the disease, infection, or disorder. Similarly, as used herein, an “effective amount” or a “therapeutically effective amount” of a compound of the disclosure refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the disclosure are outweighed by the therapeutically beneficial effects.

Phrases such as “under conditions suitable to provide” or “under conditions sufficient to yield” or the like, in the context of methods of synthesis, as used herein refer to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

The term “chemically feasible” as used herein refers to a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

The term “analog” of a chemical structure as used herein, refers to a chemical structure that preserves substantial similarity with the parent structure, although it may not be readily derived synthetically from the parent structure. A related chemical structure that is readily derived synthetically from a parent chemical structure is referred to as a “derivative.”

All chiral, diastereomeric, racemic forms of a structure are intended, unless a particular stereochemistry or isomeric form is specifically indicated. Compounds used in the present disclosure can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the disclosure.

The terms “stable compound” and “stable structure” as used herein are meant to indicate a compound that is sufficiently robust to survive isolation to a useful degree of purity from a reaction mixture, and formulation into an efficacious therapeutic agent. Only stable compounds are contemplated herein.

The term “small molecule” as used herein refers to an organic compound, including an organometallic compound, of a molecular weight less than about 2 kDa, that is not a polynucleotide, a polypeptide, a polysaccharide, or a synthetic polymer composed of a plurality of repeating units.

As to any of the groups described herein, which contain one or more substituents, it is understood that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein can be present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

The terms “amino protecting group” or “N-protected” as used herein refer to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-d imethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The terms “hydroxyl protecting group” or “O-protected” as used herein refer to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-d imethoxybenzyloxycarbonyl, 3,5-dimethoxybenzyloxycarbonyl, 2,4-d imethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

The term “substituted” as used herein refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′) 2, CN, NO, NO₂, ONO₂, azido, CF₃, OCF3, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)_{0-2N}N(R′)C(O)R′, (CH₂)_{0-2N}N(R′)N(R′)₂, N(R′)N(R′)C(O)R′, N(R′)N(R′)C(O)OR′, N(R′)N(R′)CON(R′)₂, N(R)SO₂R′, N(R)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂), or (CR′₂)ₙ wherein n is 1, 2, 3, or more, and each R′ is independently selected.

C(O) and S(O)₂ groups can also be bound to one or two heteroatoms, such as nitrogen or oxygen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a “urea.” When a C(O) is bonded to one oxygen and one nitrogen atom, the resulting group is termed a “carbamate” or “urethane.” When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

The term “ring system” as used herein refers to a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” as used herein refer to a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N-1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkyl” by itself or in combination with another term as used herein refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂CH₂—OH, —CH₂—CH₂—NH—CH₃, —CH₂—S—CH₂—CH₃, —CH₂CH₂—S(═O)—CH₃, and —CH₂CH₂—O—CH₂CH₂—O—CH₃. Up to two heteroatoms may be consecutive, such as, for example, —CH₂—NH—OCH₃, or —CH₂—CH₂—S—S—CH₃.

A “cycloheteroalkyl” ring is a cycloalkyl ring containing at least one heteroatom. A cycloheteroalkyl ring can also be termed a “heterocyclyl,” described below.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and —CH═CH—O—CH₂CH₂—O—CH₃.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl ), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl-1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The terms “aryloxy” and “arylalkoxy” as used herein refer to, respectively, an aryl group bonded to an oxygen atom and an aralkyl group bonded to the oxygen atom at the alkyl moiety. Examples include but are not limited to phenoxy, naphthyloxy, and benzyloxy.

The term “acyl” group as used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” as used herein includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino” group as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

The term “azido” as used herein refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

The term “sulfonamide” (or “sulfonamido”) as used herein includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” as used herein includes groups of the formula —C(NR)NR₂. Typically, an amidino group is —C(NH)NH₂. The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like.

A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form.

The compounds of the present disclosure may take the form of salts. The term “salts” as used herein refers to addition salts of free acids or free bases which are compounds of the disclosure. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds of the disclosure.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of the compounds of the disclosure, for example in their purification by recrystallization. All of these salts may be prepared by conventional means from the corresponding compound by reacting, for example, the appropriate acid or base with the compound.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

The term “prodrug” as used herein is well known in the art and refers to a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the disclosure are described in terms of Markush groups, those skilled in the art will recognize that the disclosure is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the disclosure are described in terms of Markush groups, those skilled in the art will recognize that the disclosure is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The present disclosure further embraces isolated compounds of the disclosure. The expression “isolated compound” refers to a preparation of a compound of the disclosure, or a mixture of compounds the disclosure, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the disclosure or a mixture of compounds of the disclosure, which contains the named compound or mixture of compounds of the disclosure in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the disclosure and intermediates may be isolated from their reaction mixtures and purified by standard techniques such as filtration, liquid-liquid extraction, solid phase extraction, distillation, recrystallization or chromatography, including flash column chromatography, or HPLC.

The term “tautomerism” as used herein refers to a compound of the disclosure or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds of the disclosure.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S).

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

The term “isolated optical isomer” as used herein refers to a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound of the disclosure, or a chiral intermediate thereof, is separated into 99% wt. % pure optical isomers by HPLC using a suitable chiral column, such as a member of the series of DAICEL® CHIRALPAK® family of columns (Deicel Chemical Industries, Ltd., Tokyo, Japan). The column is operated according to the manufacturers instructions.

The term “rotational isomerism” as used herein refers to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer.

The preferred compounds of the present disclosure have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. The term “patient” is intended to include such human and non-human mammalian species.

The pharmaceutical compositions of the subject disclosure can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the disclosure. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19th ed., 1995) describes formulations that can be used in connection with the subject disclosure.

Formulations suitable for parenteral administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject disclosure can include other agents conventional in the art having regard to the type of formulation in question.

A compounds of the disclosure such as, but not limited to formula I, or a pharmaceutically acceptable salt thereof (or a pharmaceutical compositions containing but not limited to formula I or a pharmaceutically acceptable salt thereof), can be administered to a patient by any route that results in prevention or alleviation of symptoms associated with the pathological condition. For example, as described in more detail below, but not limited to formula I or a pharmaceutically acceptable salt thereof can be administered parenterally, intravenously (i.v.), intramuscularly (i.m.), subcutaneously (s.c.), intradermally (i.d.), orally, intranasally, etc. Examples of intranasal administration can be by means of a spray, drops, powder or gel. However, other means of drug administrations are well within the scope of the present disclosure.

The pharmaceutical compositions disclosed herein may be orally administered, for example, with an inert diluent or with an assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, formula I or a pharmaceutically acceptable salt thereof may be incorporated into sustained-release preparation and formulations.

The compounds of the present disclosure, or therapeutic formulations thereof, may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases of injection, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating compounds of the present disclosure, or therapeutic formulations thereof, including a pharmaceutically acceptable salt thereof, in the required amount in the appropriate solvent with other various ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Examples of “pharmaceutically acceptable carriers” include any and all solvents, dispersion media, coatings, antibacterial and antifungal agents, isotonic and absorption delaying agents and the like. The use of such media and agents for pharmaceutical active substances is well known in the art. In one embodiment, the pharmaceutically acceptable carrier is a sterile, fluid (e.g., liquid or gas) preparation rendering the pharmaceutical composition suitable for injection or inhalation. Except insofar as any conventional media or agent is incompatible with the active ingredient, its use in the therapeutic compositions is contemplated. Supplementary active ingredients can also be incorporated into the compositions.

For oral prophylaxis, compounds of the present disclosure, or therapeutic formulations thereof, or a pharmaceutically acceptable salt thereof, may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, compounds of the present disclosure, or therapeutic formulations thereof, or a pharmaceutically acceptable salt thereof, may be incorporated into an antiseptic wash containing sodium borate, glycerin and potassium bicarbonate. Compounds of the present disclosure, or therapeutic formulations thereof, or a pharmaceutically acceptable salt thereof may also be dispersed in dentifrices, including: gels, pastes, powders and slurries. Compounds of the present disclosure, or therapeutic formulations thereof, or a pharmaceutically acceptable salt thereof may be added in a therapeutically effective amount to a paste dentifrice that may include water, binders, abrasives, flavoring agents, foaming agents, and humectants.

According to the therapeutic methods of the present disclosure, compounds of the present disclosure, or therapeutic formulations thereof, or a pharmaceutically acceptable salt thereof, is administered to a patient and dosed in accordance with good medical practice, taking into account the clinical condition of the individual patient, the site and method of administration, scheduling of administration, patient age, sex, body weight, and other factors known to medical practitioners.

As used herein, the term “additional pharmacologically active agent” refers to any agent, such as a drug, capable of having a physiologic effect (e.g., a therapeutic or prophylactic effect) on prokaryotic or eukaryotic cells, in vivo or in vitro, including, but without limitation, chemotherapeutics, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radiopharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

### Abbreviations

i.v., intravenously; i.m., intramuscularly; s.c., subcutaneously; i.d., intradermally

### Description

The present disclosure provides novel pyridine-based hydrazidones and pharmaceutically acceptable compositions comprising said hydrazidones that are advantageous for inhibiting the proliferation of a microbial population infecting or colonizing an animal or human subject. The hydrazidones of the disclosure are effective in reducing the rate of proliferation of the infective microbial population. Accordingly, such that the hydrazine can be a microbicide killing the targeted microorganism or can be a biostatic agent that arrests the growth or cell division of the microorganism but may not result in the killing of the cells. In particular, the hydrazine compounds and their corresponding pharmaceutical formulations have been found to be effective against fungi such as, but not limited to, Candida species and further has inhibitory activity against other unicellular eukaryotic parasites such as, but not limited to, Trypanosoma cruzi and Leishmania spp.

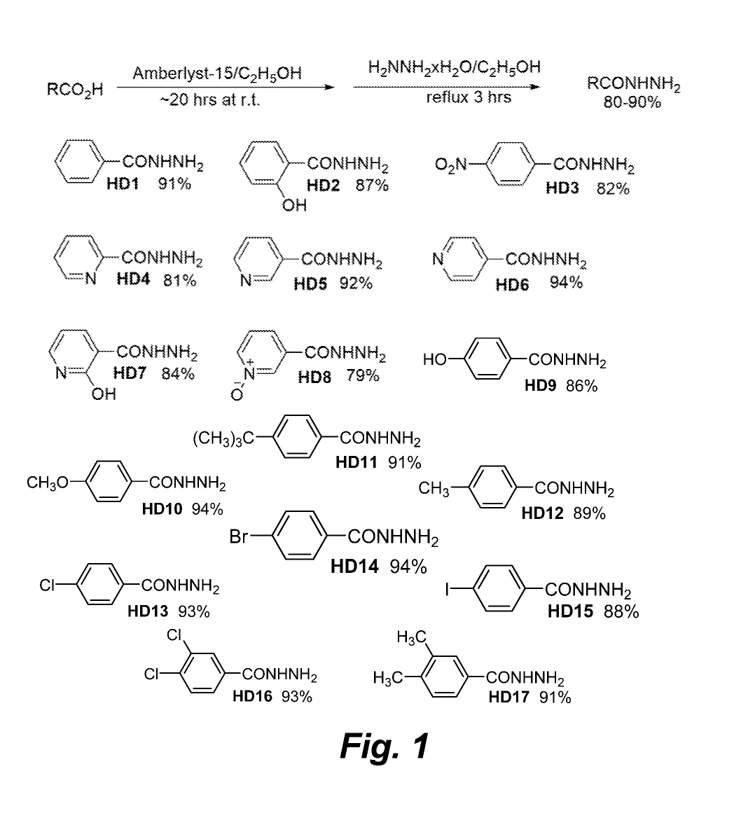

Mammalian cell toxicity assays, time-kill assays and synergy studies of the novel pyridine-based hydrazidones on both azole-susceptible and azole-resistant fungal species have shown the usefulness of the hydrazine derivatives of the disclosure for the reduction of colonizing or infecting cell populations of fungi and protistic microorganisms. The structural components that illicit potent fungal growth inhibition of both azole-susceptible and azole-resistant fungal species have been identified and a broad class of structural analogs has been synthesized. These analogs show limited toxicity against mammalian cells in vitro and exhibit fungicidal activity. Accordingly, the analogs of the present disclosure provide a useful alternative route for the development of novel antimicrobial agents.
Chemistry: All hydrazidone derivatives of the disclosure were prepared from the corresponding aromatic aldehydes and hydrazine derivatives (phenylhydrazines, hydrazides, or sulfonohydrazides). All hydrazines were prepared from the corresponding carboxylic acid through Amberlyst-15-catalyzed ethanol esterification, as previously reported (Pal et al., ARKIVOC. (2012) 570). After esterification was complete, the catalyst was removed by filtration and the filtrate was then mixed with hydrazine hydrate and refluxed for several hours to yield the desirable acid hydrazide (Scheme 1, FIG. 1). The catalyst was recyclable and it was not necessary to isolate the intermediate ester in this synthetic procedure, making the reaction essentially a one-pot reaction.

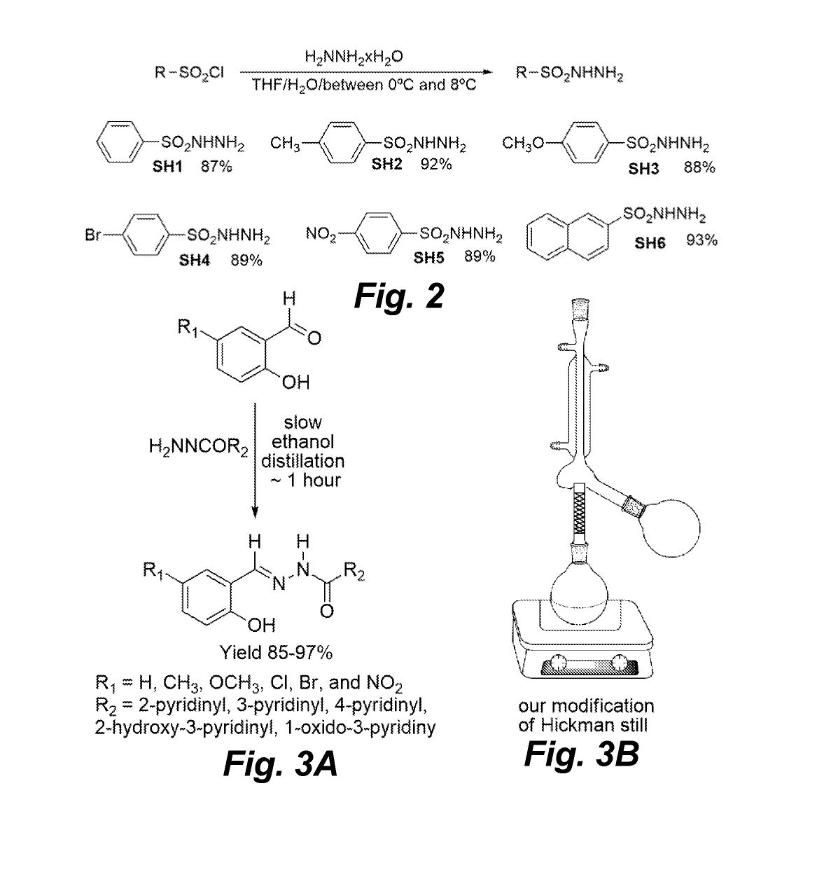

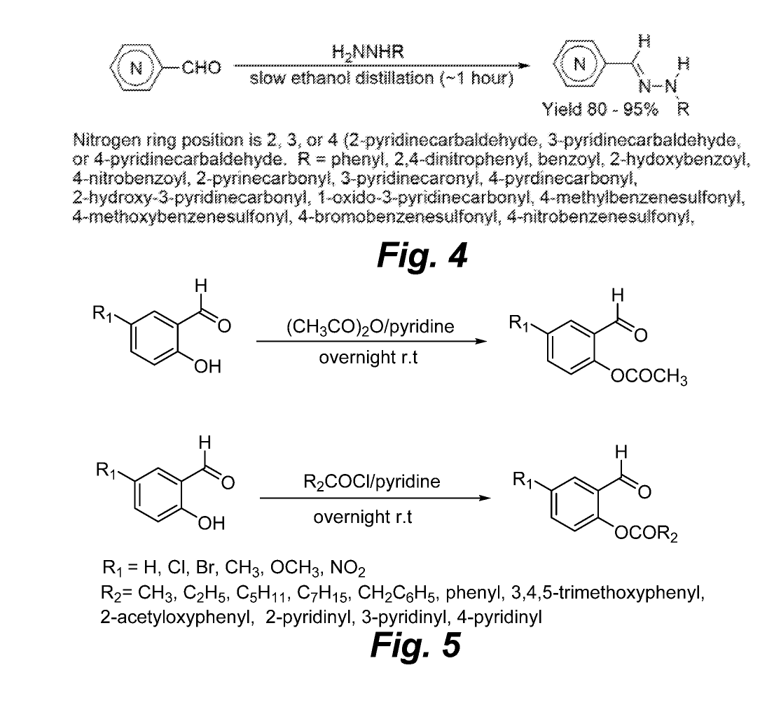

This synthetic method tolerates the presence of the phenol group and pyridine moiety as well. The sulfonohydrazides were synthesized using the large scale preparation method (Scheme 2, FIG. 2) described by Backes et al., (Bioorg. Med. Chem. (2014); 22(17):4629-4636). Hydroxybenzylidenepyridinohydrazides were prepared from 5-substituted salicylaldehyde and pyridinohydrazides in refluxing ethanol without an acid catalyst (Scheme 3, FIG. 3) (Belskaya et al., ARKIVOC. (2010) 275; Mlosto et al., Heterocycles (2014) 88: 387). This reaction was driven by increases in the reactant concentration that allowed the product to selectively precipitate from the reaction mixture. To efficiently synthesize these derivatives on a macro scale, a large scale (100 g scale in 3 L round bottom flask with 1.5 L ethanol) synthetic method was developed by using a modification of the Hickman's still head with an attached condenser and distillation receiving flask (Scheme 3, FIG. 3). A smaller reactor with a 50 ml round bottom flask was used for preparation of approximately 300 mg of individual members of the diverse pyridine-based hydrazone library. This was accomplished by slow ethanol distillation, where the rate of distillation was controlled by both the size of pre-column and the temperature of the reaction mixture. Distillation was stopped when precipitate started to form in the reaction flask. The distilled ethanol was recyclable, so only 200 ml of ethanol was needed for the preparation of more than fifty members of our hydrazone library. The same procedure was used for the preparation of individual members of the hydrazonomethylpyridine library (Scheme 4, FIG. 4).

### Antifungal Activity-Minimum Inhibitory Concentration (MIC) Studies

The antifungal activity of the pyridinohydrazide and hydrazomethylpyridine derivatives described in the s were evaluated in vitro using Candida albicans (ATCC Accession No. 10231) and Candida glabrata (ATCC Accession No. 48435). In addition, the compounds were evaluated for activity against two previously-described clinical isolates of C. albicans known to have developed azole-resistance. These isolates were obtained over a two year period from a single AIDS patient and the development of the azole-resistance mechanisms have been extensively characterized (White T C. Antimicrob. Agents Chemother. (1997) 41: 1482-1487). MIC assays were performed on the clinical isolates 1 and 17 (annotated TW1 and TW17, respectively), where isolate 1 was used as an azole-susceptible control and isolate 17 was considered azole-resistant. All assays were done in accordance with NCCLS reference documents (Clinical Laboratory and Standards Institute. Reference method for Broth Dilution Antifungal Susceptibility Testing of Yeasts: Approved Standard. M27-A3 2010; 28). The results of these screenings are summarized in Tables 1-9 (Examples 20-27) as the minimum concentrations that inhibited more than 80% of fungal growth (MIC₈₀) as compared to positive controls containing 1% DMSO. Assays were performed in HEPES-buffered RPMI-1640 media. All MIC screens used a visual scoring method as opposed to spectroscopic analysis, due to the altered absorbance spectra of many of the compounds.

### Mammalian Cell Toxicity Studies

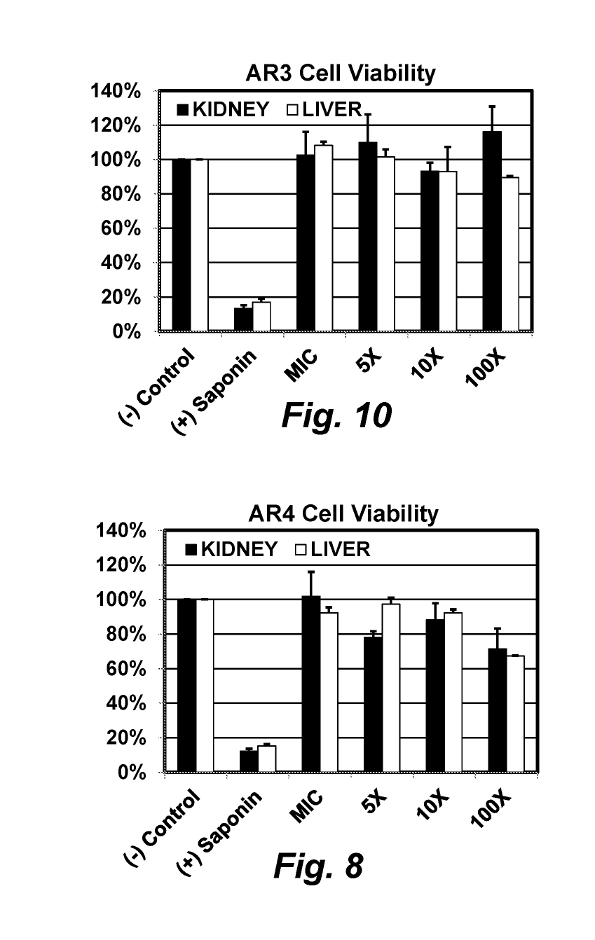

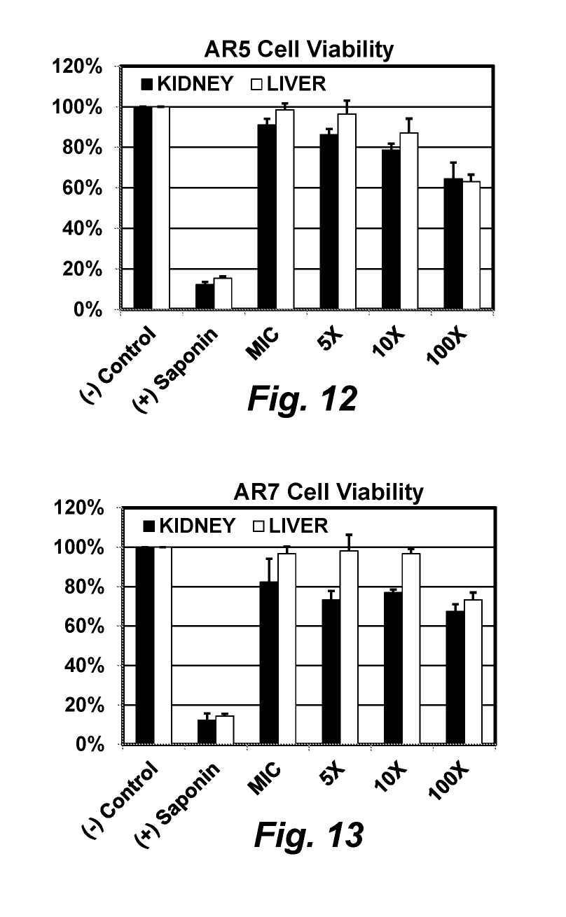

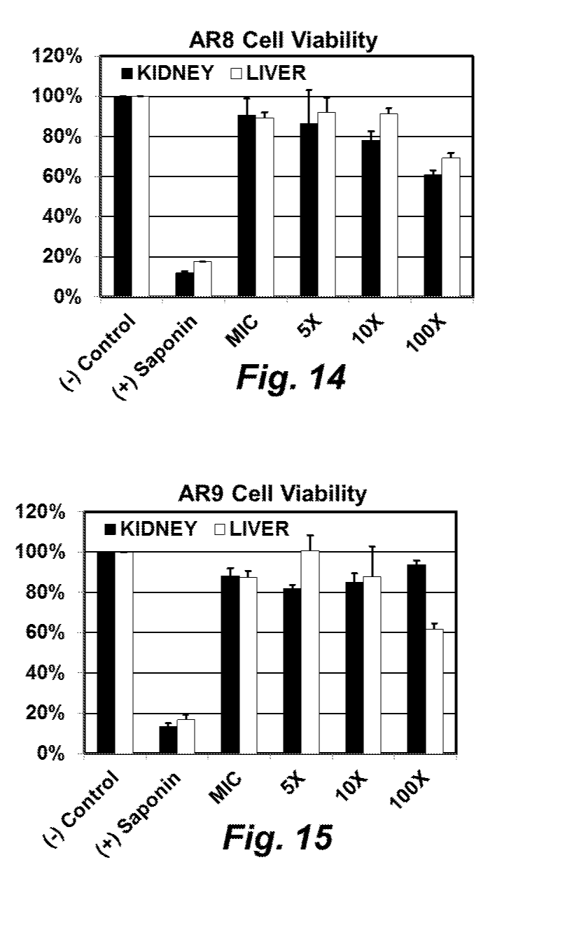

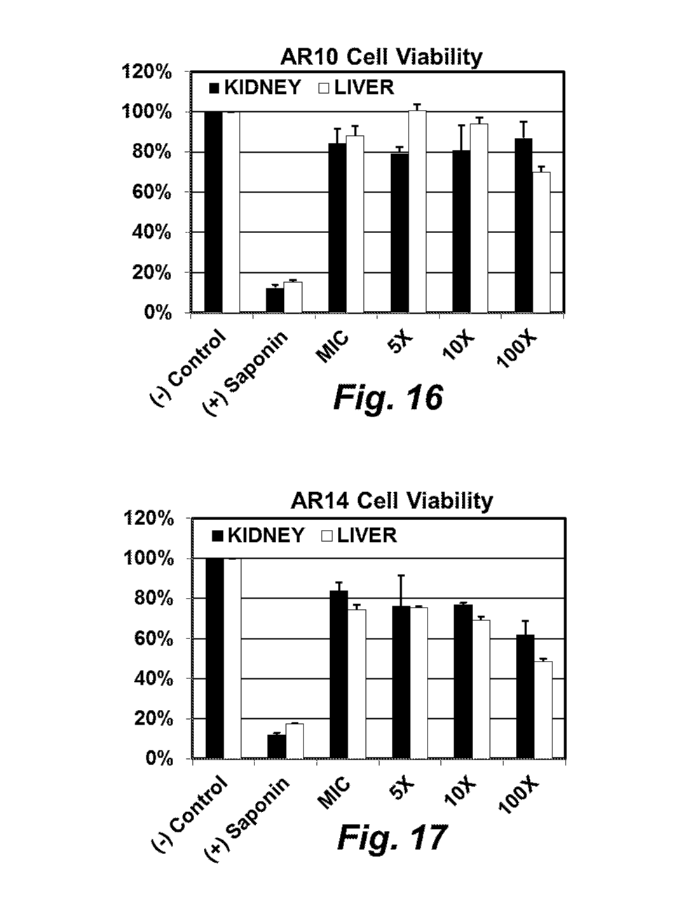

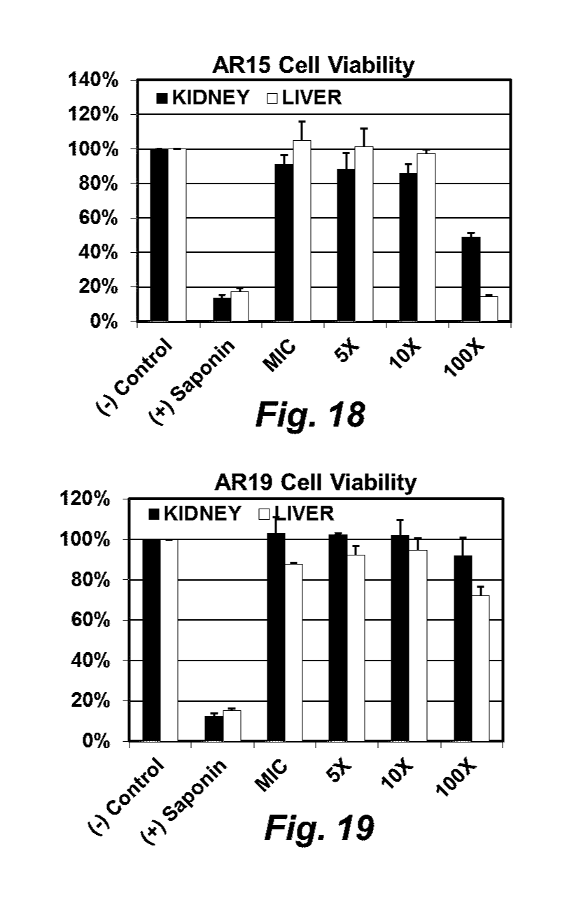

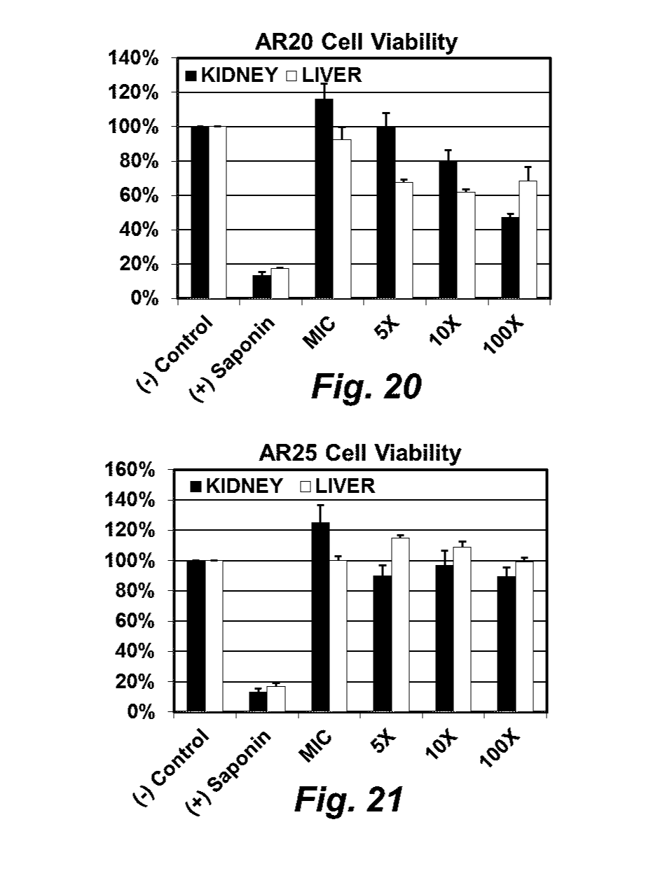

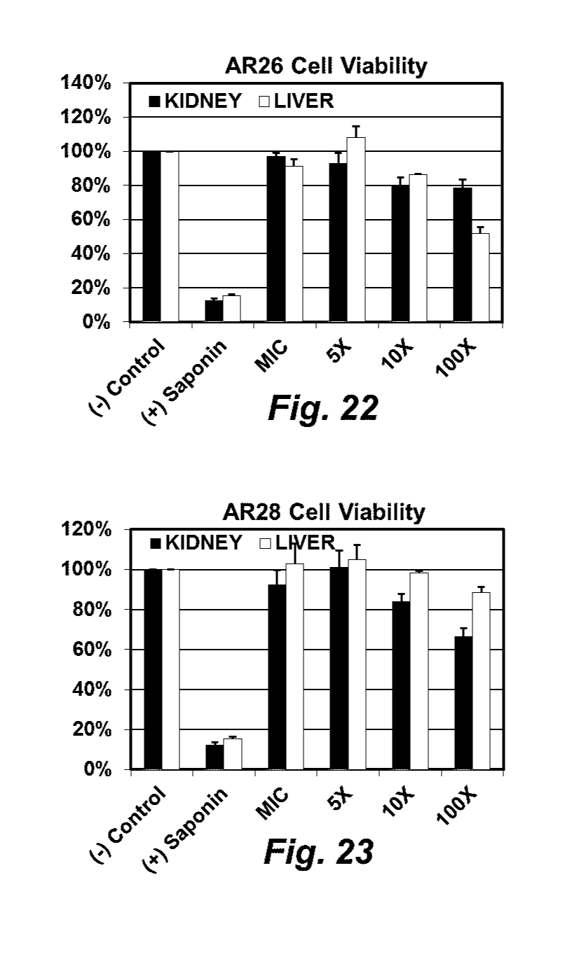

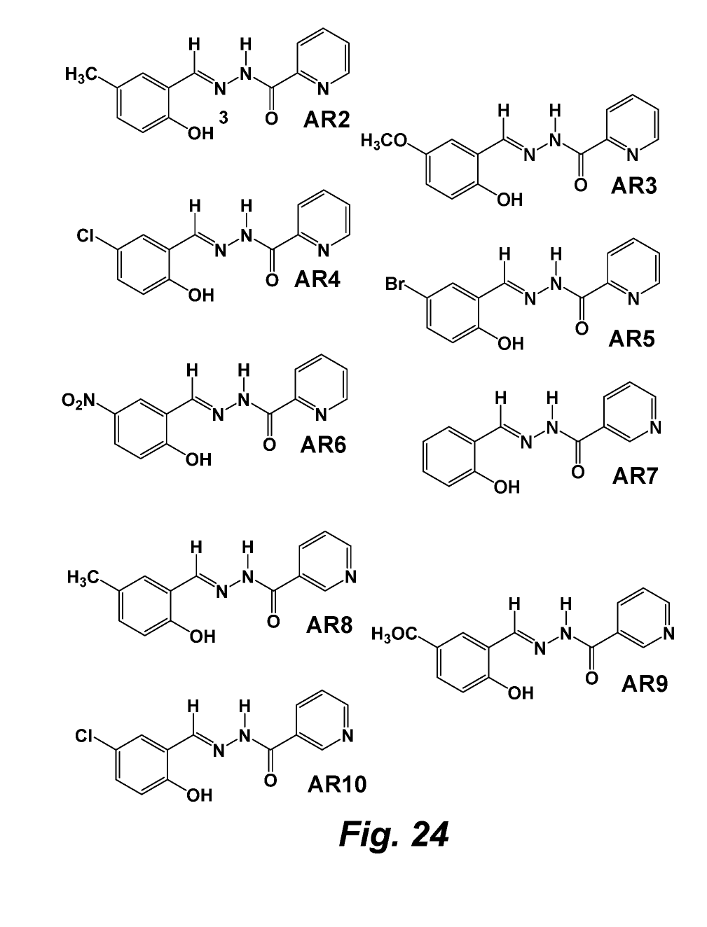

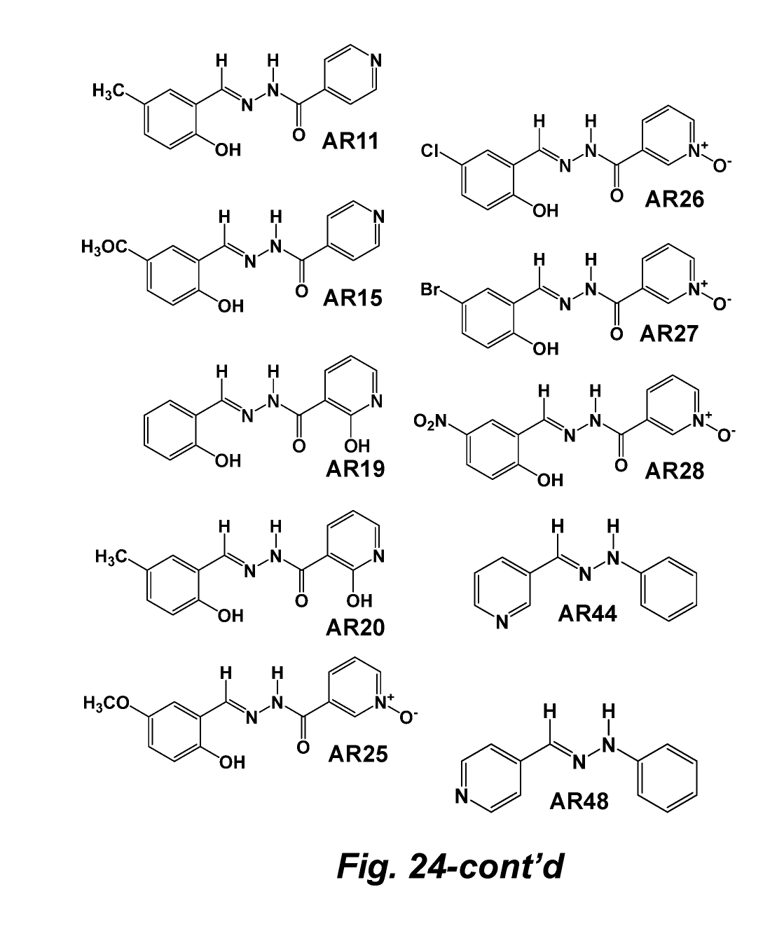

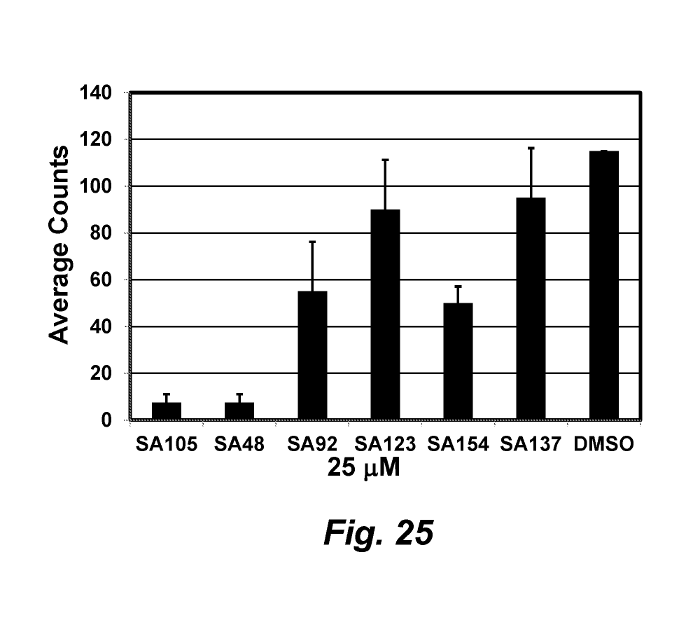

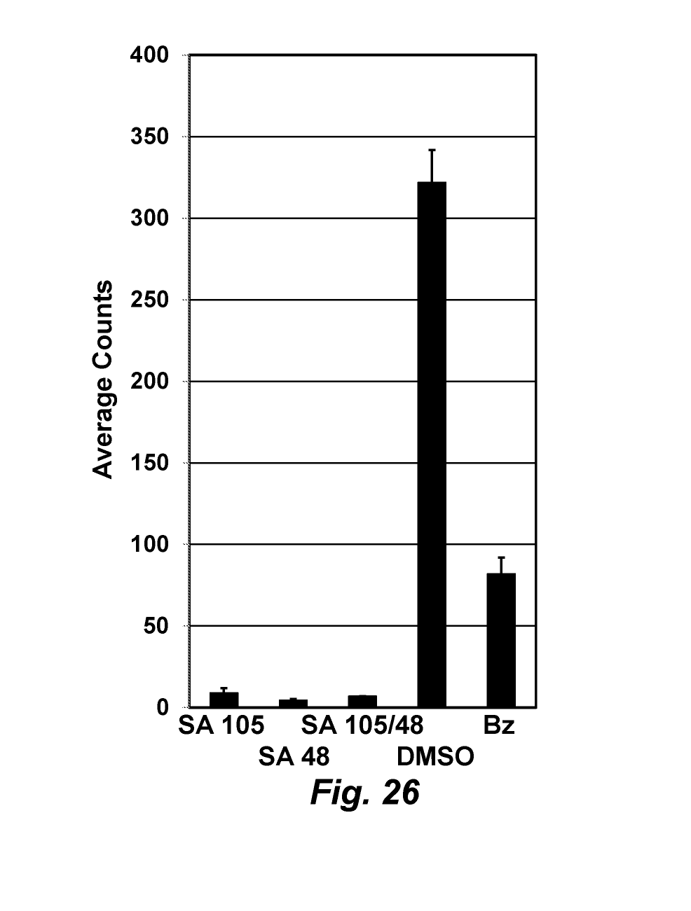

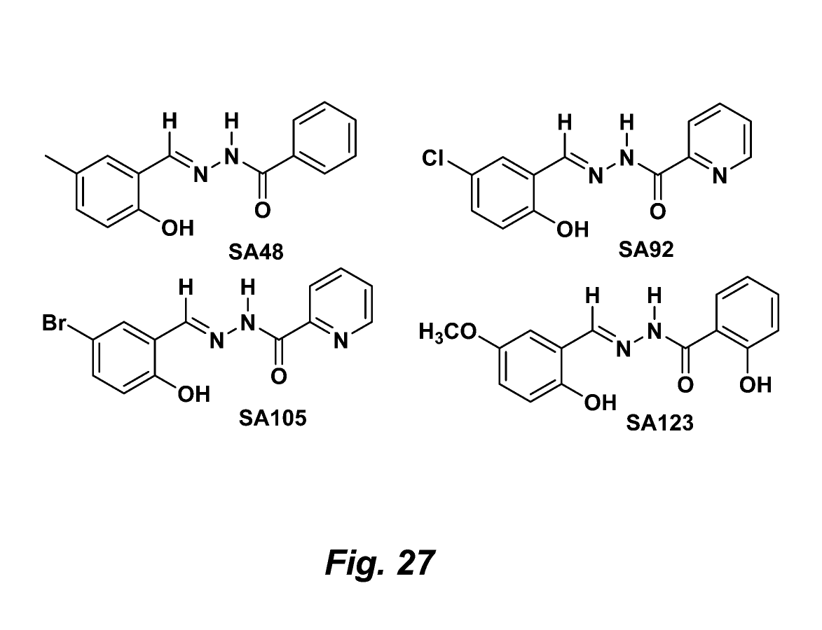

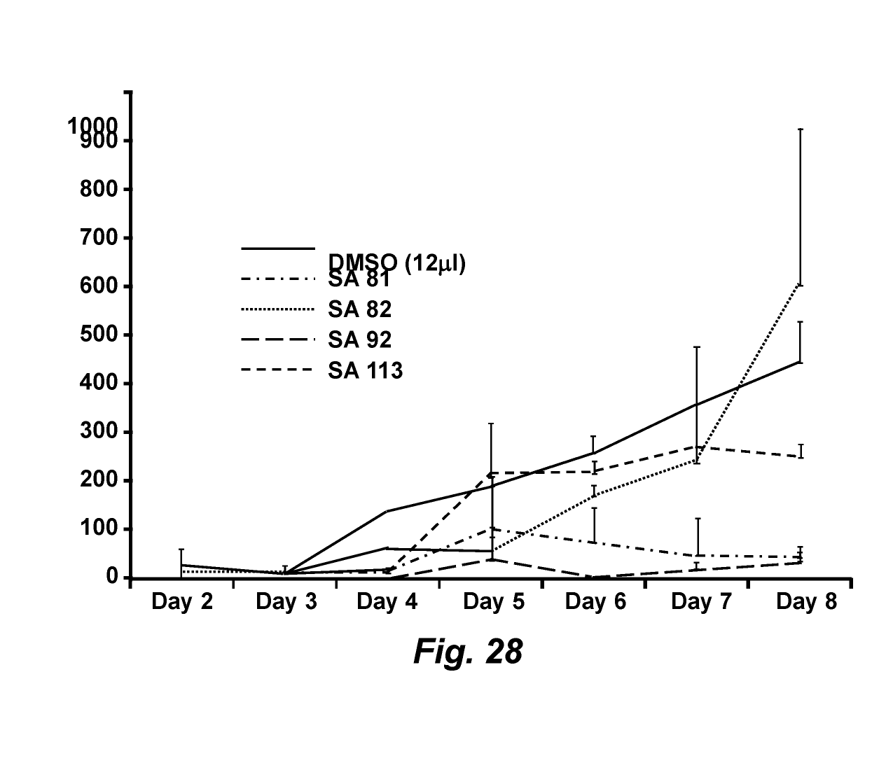

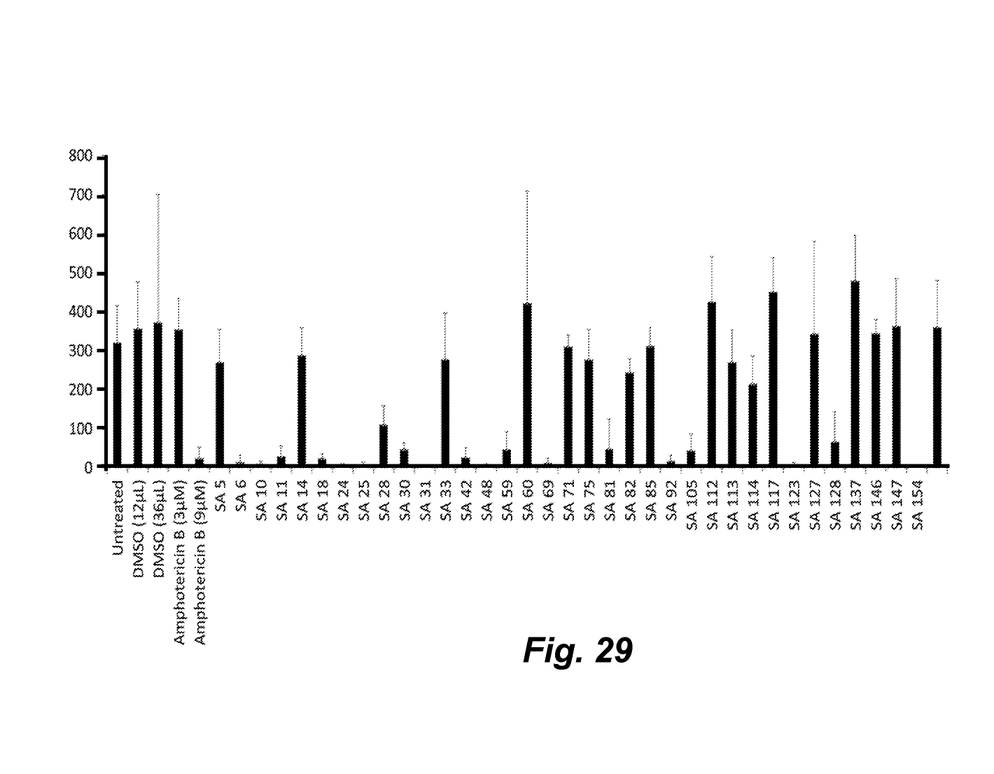

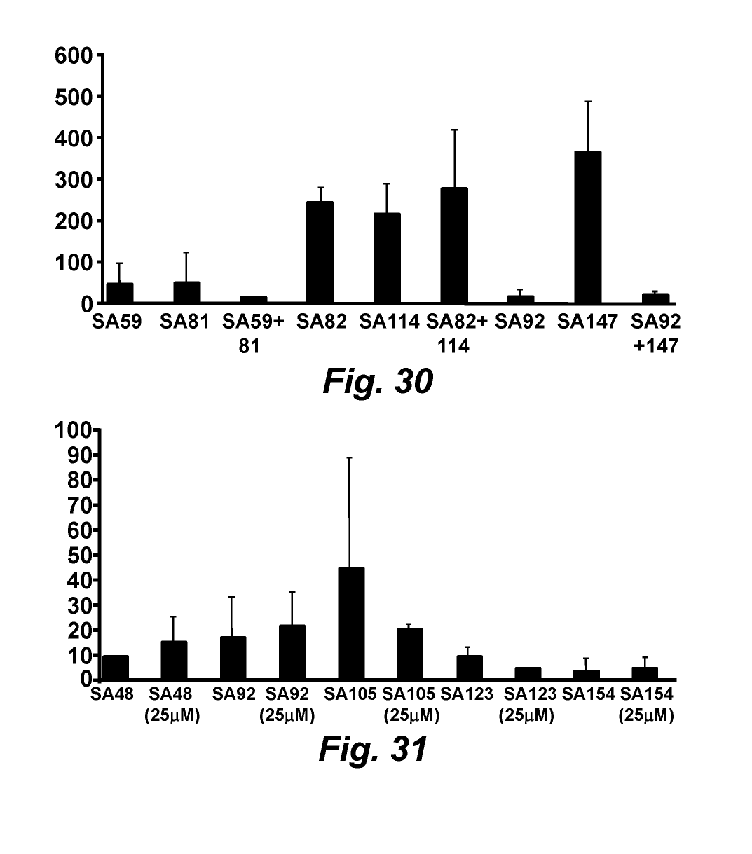

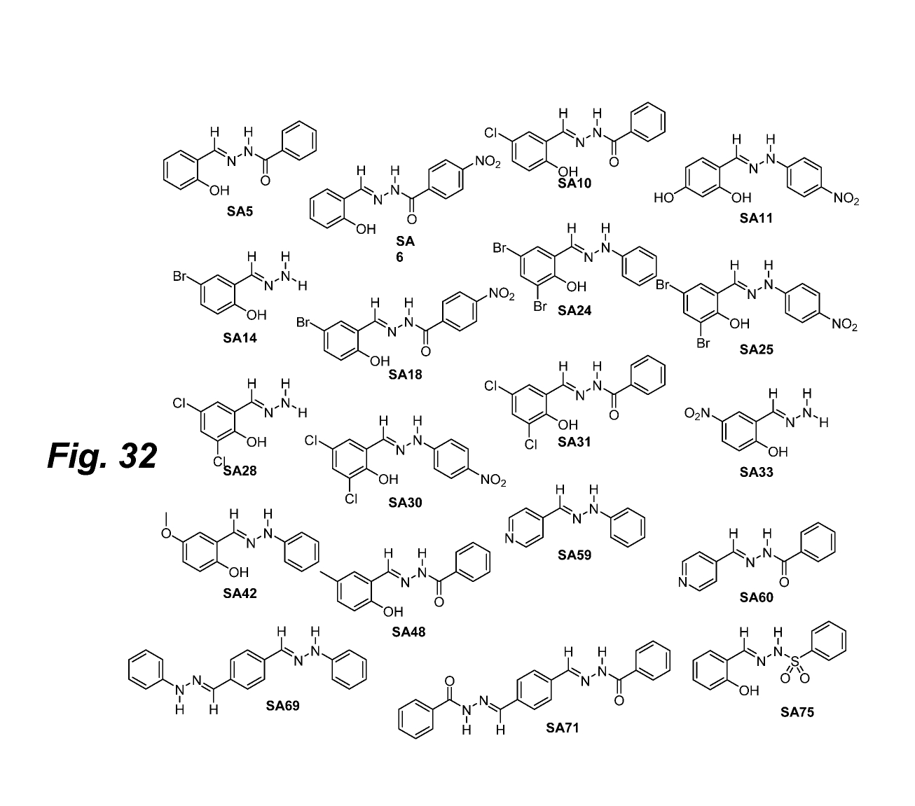

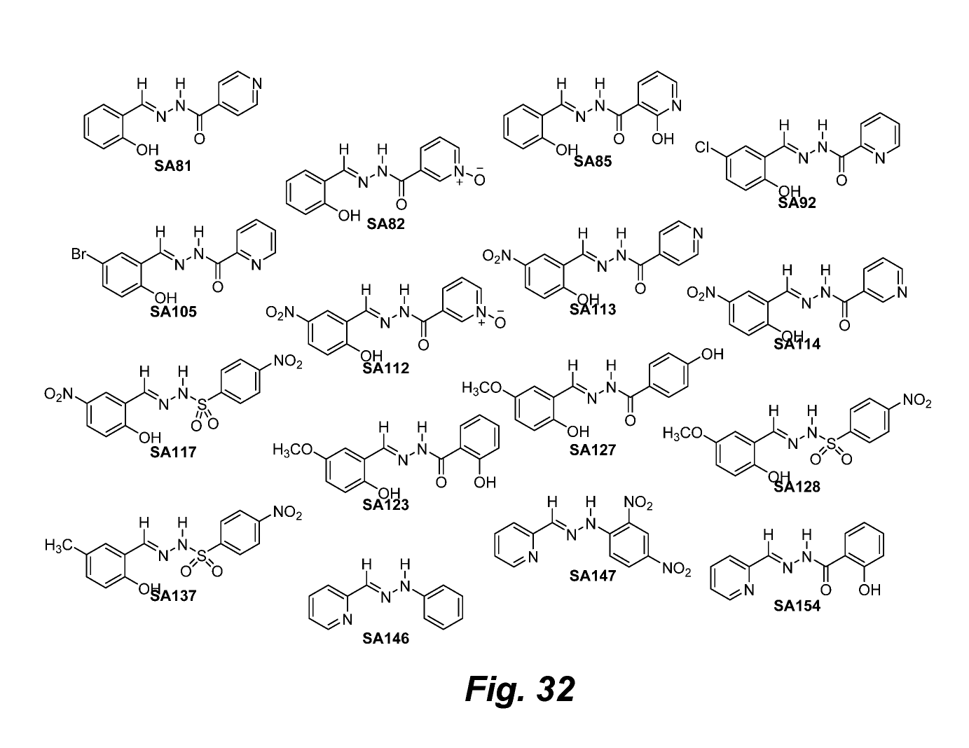

Following the fungal growth inhibition assays, compounds displaying antifungal activity at concentrations below or equal to 8 μg/mL were further subjected to in vitro mammalian cell toxicity studies using mammalian kidney cells and human liver cells (Vero (kidney) cells -ATCC Accession No. CRL-1651 and Hep G2 (liver) ATCC Accession No. HB-8065). Active analogs meeting this criterion were screened at multiple concentrations, including the lowest MIC among the Candida isolates as well as at multiples of 5, 10 and 100 times the MIC. Cytotoxicity studies were performed in accordance with Promega CellTiter 96 Non-RadioactivCell Proliferation Assay (cat #G4000). All cytotoxicity studies used a negative control (wells of either kidney or liver cells containing 1% DMSO but lacking the analog), a positive control (wells of either kidney or liver cells treated with the known cytotoxic agent, saponin) and the serial dilutions of the novel analogs. All control (negative) wells were normalized to 100% viable cells to accurately show the percentage of either liver or kidney cells that remained alive and viable following either overnight incubation with the analogs or 5-minute treatment with 0.1% saponin. Representative active compounds from each of the structural classes synthesized and tested. Results are shown in FIGS. 10-23.

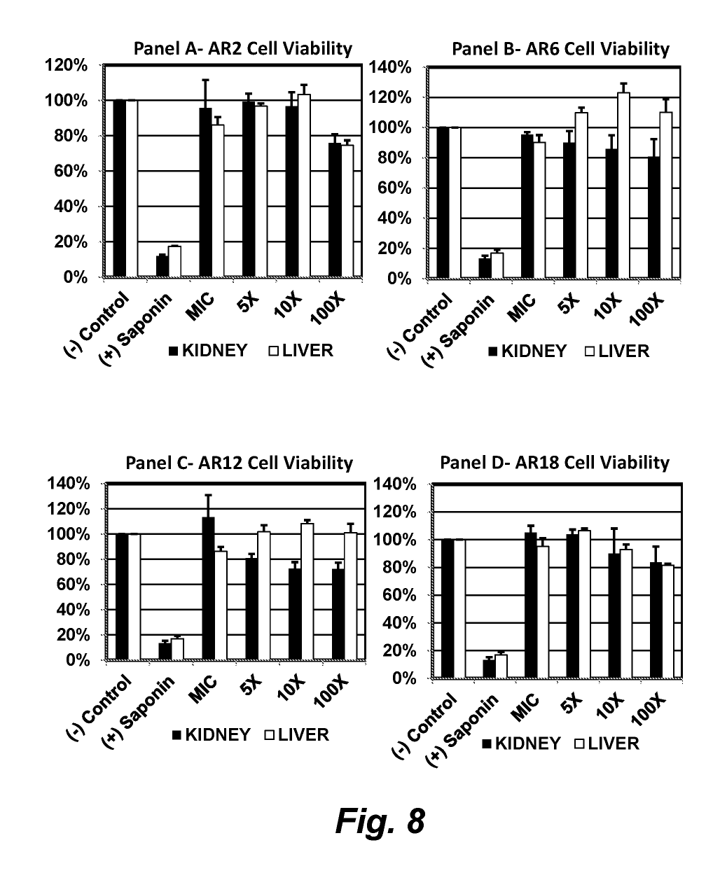

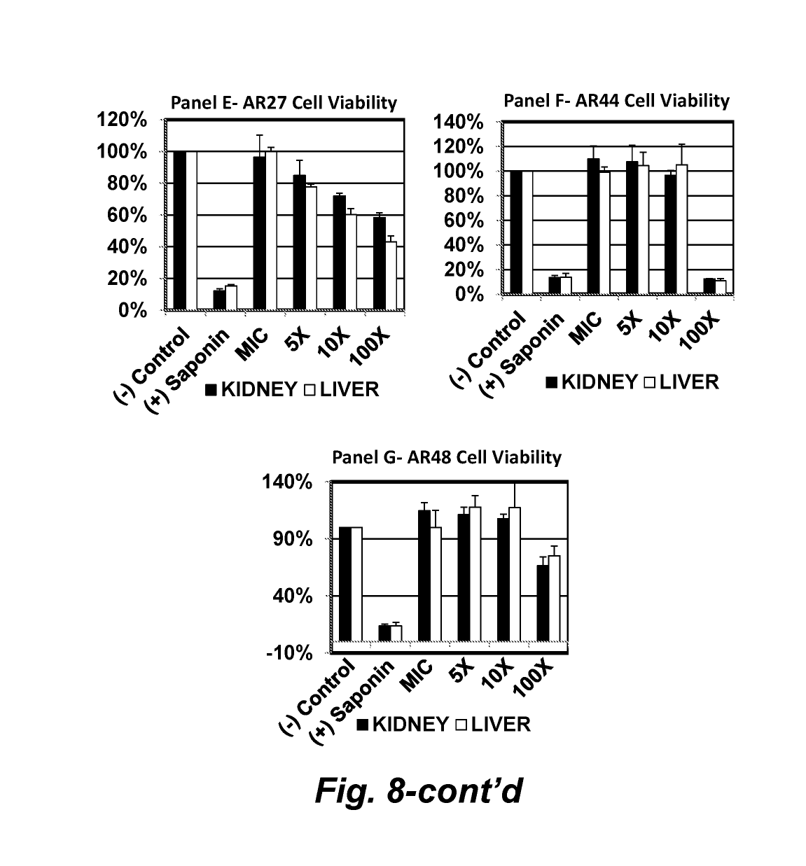

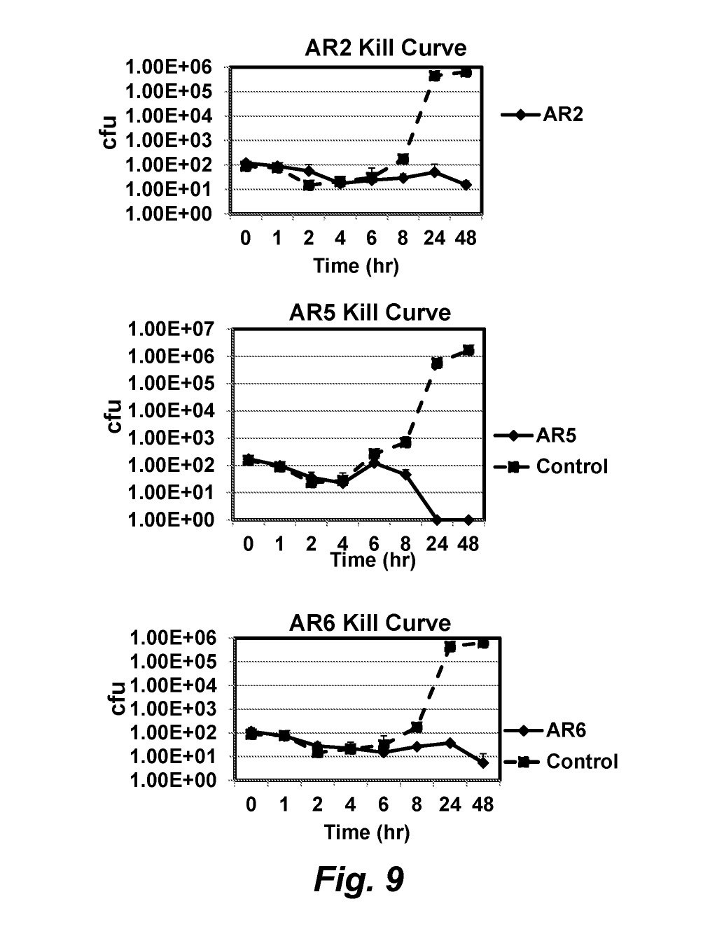

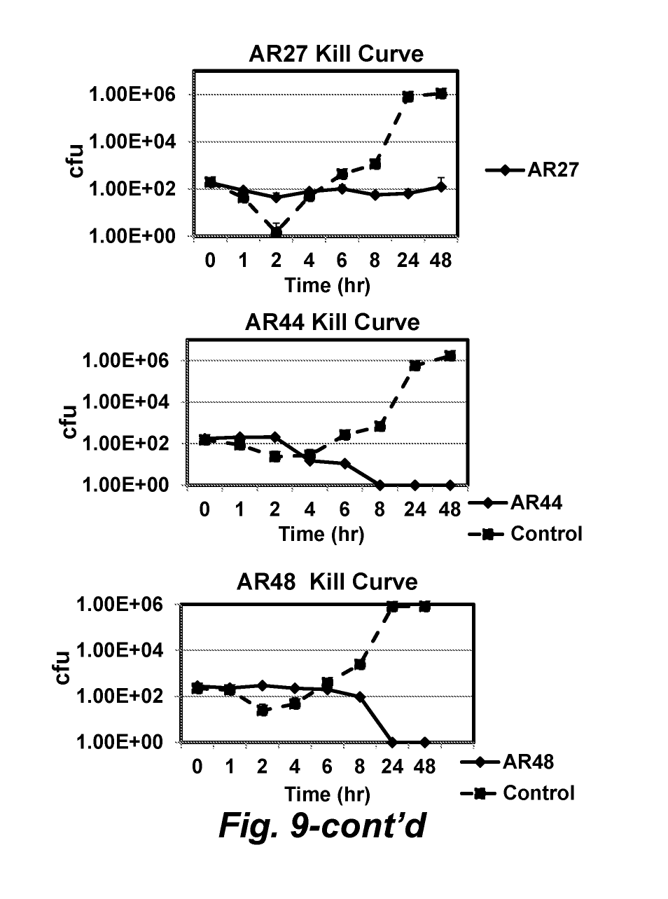

It was found that the picolinohydrazide AR2 (Table 1) showed only minimal toxicity, even at the highest concentration of 100 times greater than the MIC (FIG. 8, Panel A). Similar trends in toxicity were observed with other active compounds in Table 1 and FIGS. 8 and 9.

The nicotinohydrazides, represented by AR12 and shown in Table 2, had elevated toxicity in the kidney cells, even at the lower concentrations of 5 times the MIC (FIG. 8, Panel B). Isonicotinohydrazide AR18 (Table 3) was not toxic through 10× MIC and showed minimal cytotoxicity at the 100× MIC concentration. Similarly, the 2-hydroxy nicotinohydrazide AR19 showed minimal toxicity at concentrations through 10× MIC (FIG. 8, panels C and D).

One of the more promising pyridine oxide analogs (Table 5) was cytotoxic to liver and kidney cells, even at the 5× MIC concentrations, (AR27, FIG. 8, Panel E). Finally, the aryl hydrazone pyridines of AR44 and AR48 (Tables 8 and 9) showed no cell toxicity through 10× MIC concentrations of the MIC, with the 100× MIC concentrations being toxic to both kidney and liver cells (FIG. 8). In summary, while some toxicity was observed with a few of the analogs tested, the majority of compounds analyzed had limited cytotoxicity in the mammalian cell lines tested.

The present compounds (including pharmaceutically acceptable salt and/or hydrate forms thereof), while having biocidal or biostatic activity against Candida species, should also be contemplated as having advantageous antimicrobial (and in particular antifungal) activities against, but not limited to, a wide range of yeasts and fungi and even unicellular protozoa, including one or more of the following: Acremonium, Absidia (e.g., Absidia corymbifera), Alternaria, Aspergillus (e.g., Asp. clavatus, Asp. flavus, Asp. fumigatus, Asp. nidulans, Asp. niger, Asp. terreus, and Asp. versicolor), Bipolaris, Blastomyces (e.g., Blastomyces dermatitidis), Blastoschizomyces (e.g., Blastoschizomyces capitatus), C. (e.g., C. albicans, C. glabrata (Torulopsis glabrata), C. guilliermondii, C. kefyr, C. krusei, C. lusitaniae, C. parapsilosis, C. pseudotropicalis, C. stellatoidea, C. tropicalis, C. utilis, C. lipolytica, C. famata and C. rugosa), Cladosporium (e.g., Cladosporium carrionii and Cladosporium trichloides), Coccidioides (e.g., Coccidioides immitis), Cryptococcus (e.g., Cryptococcus neoformans), Curvularia, Cunninghamella (e.g., Cunninghamella elegans), Dermatophyte, Exophiala (e.g., Exophiala dermatitidis and Exophiala spinifera), Epidermophyton (e.g., Epidermophyton floccosum), Fonsecaea (e.g., Fonsecaea pedroso), Fusarium (e.g., Fusarium solan), Geotrichum (e.g., Geotrichum candiddum and Geotrichum clavatum), Histoplasma (e.g., Histoplasma capsulatum var. capsulatum), Malassezia (e.g., Malassezia furfur), Microsporum (e.g., Microsporum canis and Microsporum gypseum), Mucor, Paracoccidioides (e.g., Paracoccidioides brasiliensis), Penicillium (e.g., Penicillium marneffe), Phialophora, Pityrosporum ovale, Pneumocystis (e.g., Pneumocystis carini), Pseudallescheria (e.g., Pseudallescheria boydi), Rhizopus (e.g., Rhizopus microsporus var. rhizopodiformis and Rhizopus oryzae), Saccharomyces (e.g., Saccharomyces cerevisiae), Scedosporium (e.g., Scedosporium apiosperum), Scopulariopsis, Sporothrix (e.g., Sporothrix schencki), Trichoderma, Trichophyton (e.g., Trichophyton mentagrophytes and Trichophyton rubrum), and Trichosporon (e.g., Trichosporon asahii, Trichosporon beigelii and Trichosporon cutaneum).

The present compounds are believed to be not only useful against organisms causing systemic human pathogenic mycotic infections or colonizations, but also useful against organisms causing superficial infections such as Trichoderma sp. and other Candida. spp. The compounds of the present disclosure are believed to be particularly effective against Asp. flavus, Asp. fumigatus, C. albicans, C. parapsilosis, Cryptococcus neoformans, Saccharomyces cerevisiae, and Trichophyton mentagrophytes.

In view of their antifungal activity, compounds as disclosed are considered useful for the treatment and/or prevention of one or more of a variety of superficial, cutaneous, subcutaneous and systemic mycotic infections in skin, eye, hair, nail, oral mucosa, gastrointestinal tract, bronchus, lung, endocardium, brain, meninges, urinary organ, vaginal portion, oral cavity, ophthalmus, systemic, kidney, bronchus, heart, external auditory canal, bone, nasal cavity, paranasal cavity, spleen, liver, hypodermal tissue, lymph duct, gastrointestine, articulation, muscle, tendon, interstitial plasma cell in lung, blood, and so on.

Therefore, compounds of the present disclosure are useful for preventing and treating one or more of various infectious diseases, such as dermatophytosis (e.g., trichophytosis, ringworm or tinea infections), athletes foot, paronychia, pityriasis versicolor, erythrasma, intertrigo, fungal diaper rash, candida vulvitis, candida balanitis, otitis externa, candidiasis (cutaneous and mucocutaneous), chronic mucocandidiasis (e.g. thrush and vaginal candidiasis), cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, and fungemia. The present compounds may also be used as prophylactic agents to prevent systemic and topical fungal infections. Use as prophylactic agents may, for example, be appropriate as part of a selective gut decontamination regimen in the prevention of infection in immuno-compromised patients (e.g. AIDS patients, patients receiving cancer therapy or transplant patients). Prevention of fungal overgrowth during antibiotic treatment may also be desirable in some disease syndromes or iatrogenic states.

It is further contemplated that the compounds of the present disclosure may be mixed with other antifungal agents and may have either a synergistic or additive effect against the target fungal species. For example, but not intended to be limiting, examples of azoles that may be used in combination with the present compounds include, but are not limited to, fluconazole, voriconazole, itraconazole, ketoconazole, miconazole, ravuconazole, detoconazole, clotrimazole, and posaconazole. Examples of polyenes that may be used in combination with the present compounds include, but are not limited to, amphotericin B, nystatin, liposamal and lipid forms thereof such as ABELCET, AMBISOME, and AMPHOCIL. Examples of purine or pyrimidine nucleotide inhibitors that may be used in combination with the present compounds include, but are not limited to, flucytosine or polyxins such as nikkomycines, in particular nikkomycine Z or nikkomycine X. Another class of therapeutic agents that may be used in combination with the present compounds includes chitin inhibitors. Examples of elongation factor inhibitors that may be used in combination with the present compounds include, but are not limited to, sordarin and analogs thereof. Examples of pneumocandin or echinocandin derivatives that may be used in combination with the present compounds include, but are not limited to, cilofungin, anidulafungin, micafungin, and caspofungin. Examples of mannan inhibitors that may be used in combination with the present compounds include but are not limited to predamycin.

The pharmaceutical composition of the present disclosure comprises at least one of the compounds of the present disclosure or a pharmaceutically acceptable salt thereof as an active ingredient. The pharmaceutical composition of the present disclosure may comprise at least one compound of the present disclosure or a pharmaceutically acceptable salt thereof, and at least one pharmaceutically acceptable carrier. The pharmaceutical composition of the present disclosure, which comprises the compound of the present disclosure or a pharmaceutically acceptable salt thereof as an active ingredient, can be used for the purpose of preventing and/or treating a fungal infection or colonization of an animal or human subject or a protozoal infection of an animal or human such as but not limited to leishmaniasis, and can be administered via parenteral administration such as intravenous administration, subcutaneous administration or intrarectal administration, as well as via oral administration, transmucosal administration or transdermal administration or the like. A variety of dosage forms suitable for these administration pathways will be well known to those skilled in the art, and a person skilled in the art could select an appropriate dosage form suitable for the desired form of administration, and then produce a formulation of the pharmaceutical composition, where necessary using one or more pharmaceutically acceptable carriers or formulation additives that can be used within the field. For example, in the case of transdermal administration, an ointment or other form of applied formulation, or a poultice or other form of patch is ideal. In some cases, a hydrate or solvate of the compound of the present disclosure or pharmaceutically acceptable salt thereof may be used as the active ingredient of the medicine of the present disclosure. There are no particular limitations on the dose, and for example, a single dose may be set within a range from 0.1 to 10 mg in the case of transdermal administration or transmucosal administration, or within a range from 1 to 100 mg in the case of an oral administration, with administration being performed 2 or 3 times per day. Alternatively, a dose of approximately 0.1 to 1,000 mg, and preferably approximately 1 to 300 mg, may be typically administered per day to an adult. Further, the dose can also be set in relation to parameters such as the weight, age, genetic type and symptoms of the patient.

The pharmaceutical compositions of the present disclosure may be provided in dosage forms such as tablets, granules (fine grains), capsules, injections (intravenous drips), patches, suppositories, suspensions and emulsions, pastes, ointments, creams, lotions, nasal drops and eye drops, but not limited to them. In some cases, the pharmaceutical composition of the present disclosure may be converted to a controlled-release form in order to enable sustained release over a long period of time.

The pharmaceutically acceptable carriers or formulation additives included in the pharmaceutical composition of the present disclosure may include stabilizers, surfactants, solubilizers and adsorbents and the like, but not limited to them. The pharmaceutically acceptable carriers or formulation additives included in the pharmaceutical composition of the present disclosure are selected in accordance with the dosage form of the pharmaceutical composition of the present disclosure described above.

### Time-Kill Assay

One of the objectives of this study was to determine the cidal or static nature of the analogs that were active against such as TW17, the azole-resistant clinical isolate. Azoles affect Candida spp. by inhibiting ergosterol biosynthesis, and ultimately prevent their reproduction. This effect has led to the classification of azoles as fungistats. Determining the fungistatic or fungicidal nature of the synthesized compounds of the present disclosure allows a comparison to azoles, in addition to improving the understanding of how the structural motifs might lend toward an alternate mechanism of action. Therefore, time-kill assays were performed using the representative analogs that were active against the azole-resistant TW17 at concentrations at or below 8 μg/mL.

Solid drugs were dissolved in DMSO to a concentration of 10 mg/ml. This stock solution was then further diluted in water to the desired experimental concentration, determined to be four times that of the MIC for the Candida strain TW17 (as reported in the Tables).

Individual colonies of TW17 were suspended in 5 ml of sterile water and adjusted to achieve a McFarland reading between 0.08 and 0.12. The inoculum was then further diluted in sterile water at a ratio of 1:100. Three 15-ml tissue culture tubes were prepared by combining 2.5 ml of above diluted inoculum, 21.7 ml of HEPES buffered RPMI-1640 media, and 800 μl of experimental drug dilution. A “Growth Control” tube was prepared by combining 2.5 ml of inoculum, 21.7 HEPES buffered RPMI-1640 media, and 800 μl of water-DMSO solution equal to the DMSO concentration of the experimental drug dilution. A “Sterile Control” tube was prepared by combining 24.2 ml of HEPES-buffered RPMI-1640 media and 800 μl of water-DMSO solution as in the “Growth Control”. All tubes were incubated for 48 hours at 35° C., 50 rpm. 500 μl samples were taken from the tubes at time points of predetermined intervals during this incubation.

Time points for the “Kill-Curve” and “Growth Control” tubes were taken at 0, 1, 2, 4, 6, 8, 24, and 48 h after the start of incubation. Time points for “Sterile Control” were taken at 0 and 48 hours. Each time point sample was serially diluted 1:10 in sterile 0.9% NaCl solution up to 5 times. 100 μl of each sample was spread onto YM agar plates with a sterile bent spreading rod. Plates were incubated at 35° C. overnight and colonies were then counted on each plate. Colonies on plates from diluted time points were used to estimate uncountable undiluted samples. The number of colonies in undiluted samples was estimated to be 10ⁿ times greater than the diluted sample, where n was the number of dilutions made.

From the time-kill assays, several compounds were identified that were fungistatic. In general, analogs with the picolinohydrazide structure (FIG. 9), the nicotinohydrazide structure and the isonicotinohydrazide structure were fungistatic and no increase in fungal growth could be observed after 48 h in the presence of these analogs.

An exception to this generalization regards the presence of a chloro or bromo substituent in the 4-position of the salicylaldehyde moiety; these analogs showed fungicidal activity (see FIG. 9, Panel B, AR5). With respect to the active pyridine oxide compound AR27 (Table 5), the presence of the bromo substituent did not render the analog fungicidal; this compound instead showed fungistatic activity. Advantageously, the aryl substituted pyridines lacking a salicylaldehyde moiety, AR44 and AR48 (Tables 8 and 9) were fungicidal, with no observed colonies after 24 h.

Accordingly, one aspect of the disclosure encompasses embodiments of a compound having the formula I, II, or III, or a hydrate or pharmaceutically acceptable salt thereof:

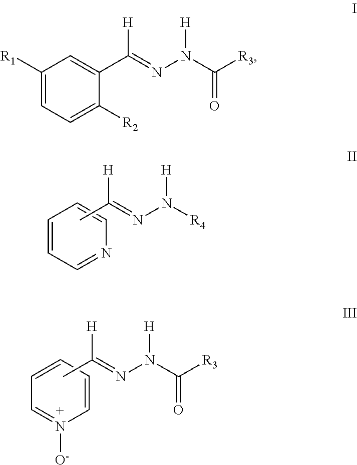

wherein in formula I: R₁ can be H, alkyl, aryl, substituted aryl, alkyloxy, substituted alkyloxy, aryloxy, substituted aryloxy, a halogen, or NO₂; R₂ can be a OH, or an ester group; R₃ can be a pyridine, a substituted pyridine, an N-oxypyridine, an aryl group, or a substituted aryl group, with the proviso that when R₂ is OH and R₃ is 2-pyridinyl then R₁ is not H, a methyl, or NO₂, and when R₂ is OH and R₃ is 3-pyridinyl or 4-pyridinyl then R₁ is not H, a methyl, a methoxy, a halogen, or NO₂; and wherein in formula II R₄ can be a phenyl, a 2,4-dinitrophenyl, a benzoyl, a 2-hydroxybenzoyl, a 4-nitrobenzoyl, a pyridine-2-carbonyl, a pyridine-3-carbonyl, a pyridine-4-carbonyl, a 2-hydroxynicotinoyl, a 1-oxidopyridine-3-carbonyl, or —(O═S═O)—R₅; and R₅ is a 2-naphthyl, a 4-methylphenyl, a 4-methoxyphenyl, a 4-bromophenyl, or a 4-nitrophenyl, and wherein in formula III R₃ can be a pyridine, a substituted pyridine, an N-oxypyridine, an aryl group, or a substituted aryl group.

In some embodiments of this aspect of the disclosure, R₂ can be an ester having the formula OCOX, wherein X is selected from the group consisting of: methyl, ethyl, cyclopentane, cycloheptane, CH₂C₆H₅, phenyl, 3,4,5-trimethoxyphenyl, 2-acetyloxyphenyl, 2-pyridinyl, 3-pyridinyl, and 4-pyridinyl.

In some embodiments of this aspect of the disclosure, R₃ can be selected from the group consisting of: phenyl, 4-methylphenyl, 4-methoxyphenyl, 4-t-butylphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3-pyridinyl, 4-pyridinyl, 1-oxidopyridin-2-yl, 1-oxidopyridin-3-yl, and 1-oxidopyridin-4-yl.

In some embodiments of this aspect of the disclosure, the compound can have the formula I

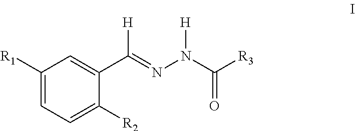

wherein: R₁ is H, an alkyl, an alkyloxy, a halogen, or NO₂; R₂ is OH, or an ester group; R₃ is a pyridine, a substituted pyridine, an N-oxypyridine, an aryl group, or a substituted aryl group, with the proviso that when R₂ is OH and R₃ is 2-pyridinyl then R₁ is not H, a methyl, or NO₂, and when R₂ is OH and R₃ is 3-pyridinyl or 4-pyridinyl then R₁ is not H, a methyl, a methoxy, a halogen, or NO₂.

In some embodiments of this aspect of the disclosure, R₂ can be an ester having the formula OCOX, wherein X is selected from the group consisting of: methyl, ethyl, cyclopentane, cycloheptane, CH₂C₆H₅, phenyl, 3,4,5-trimethoxyphenyl, 2-acetyloxyphenyl, 2-pyridinyl, 3-pyridinyl, and 4-pyridinyl.

In some embodiments of this aspect of the disclosure, R₃ can be selected from the group consisting of: phenyl, 4-methylphenyl, 4-methoxyphenyl, 4-t-butylphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3-pyridinyl, 4-pyridinyl, 1-oxidopyridin-2-yl, 1-oxidopyridin-3-yl, and 1-oxidopyridin-4-yl.

In some embodiments of this aspect of the disclosure, the compound can have the formula I, and wherein R₁ is H, CH₃, OCH₃, Cl, Br, or NO₂ and R₂ is OCOCH₃, OCOC₆H₅, or OCOC₆H₅-o-OCOCH₃.

In some embodiments of this aspect of the disclosure, the compound can have the formula II:

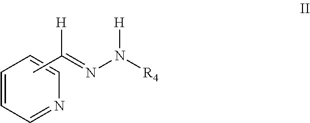

wherein: R₄ can be a phenyl, 4-methylphenyl, 4-methoxyphenyl, 4-t-butylphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3-pyridinyl, 4-pyridinyl, 1-oxidopyridin-2-yl, 1-oxidopyridin-3-yl, 1-oxidopyridin-4-yl, or —(O═S═O)—R₅; and R₅ can be a 2-naphthyl, a 4-methylphenyl, a 4-methoxyphenyl, a 4-bromophenyl, or a 4-nitrophenyl.

In some embodiments of this aspect of the disclosure the compound can have the formula III:

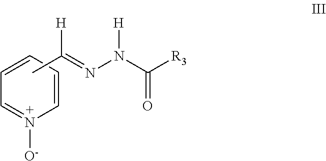

wherein R₃ can be selected from the group consisting of: phenyl, 4-methylphenyl, 4-methoxyphenyl, 4-t-butylphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3-pyridinyl, 4-pyridinyl, 1-oxidopyridin-2-yl, 1-oxidopyridin-3-yl, and 1-oxidopyridin-4-yl

In some embodiments of this aspect of the disclosure, said compound can be selected from the group consisting of

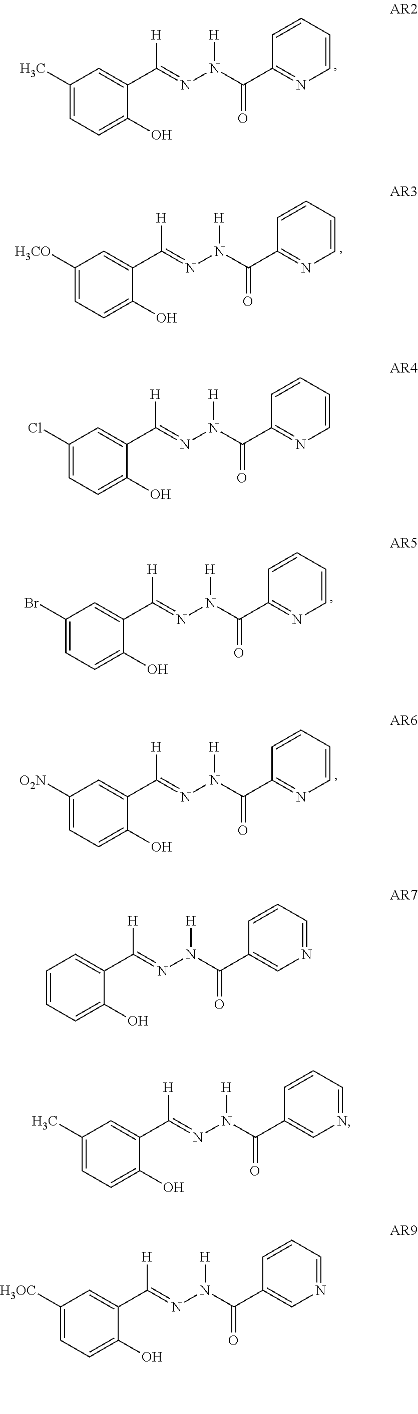

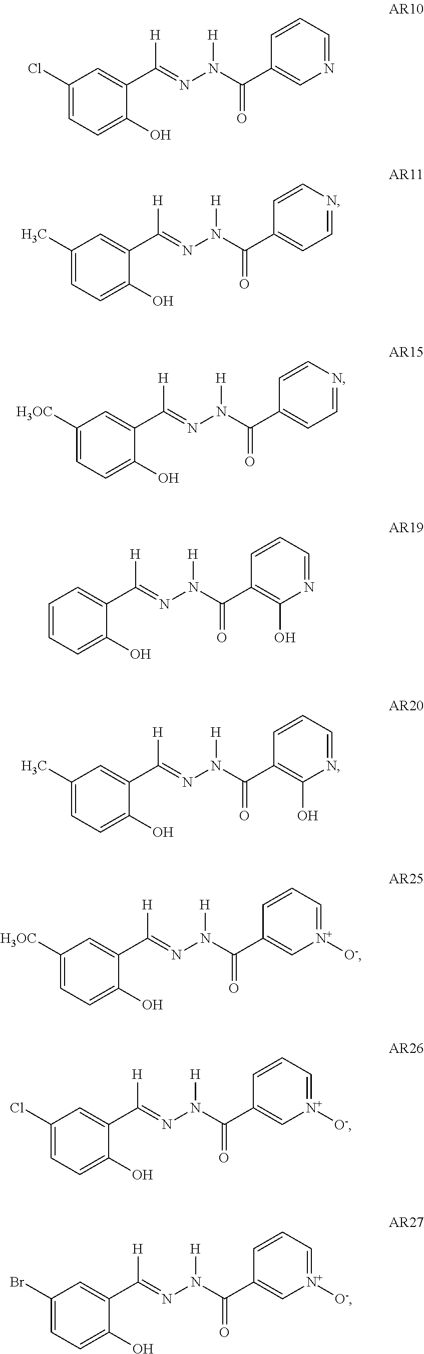

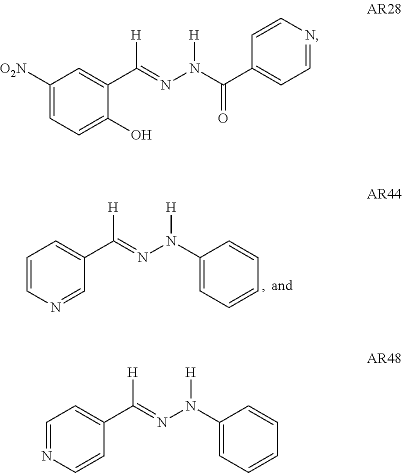 [SMILES: O=C(N/N=C/c1cc([N+](=O)[O-])ccc1O)c1ccncc1 ; C(=N\Nc1ccccc1)/c1cccnc1 ; C(=N\Nc1ccccc1)/c1ccncc1]

Another aspect of the disclosure encompasses embodiments of a pharmaceutically acceptable composition comprising at least one compound having the formula I, II, or III, or a hydrate or pharmaceutically acceptable salt thereof:

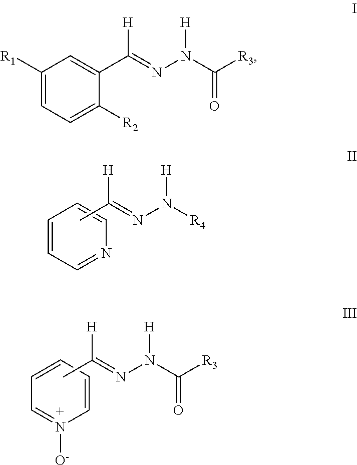

wherein in formula I: R₁ can be H, alkyl, aryl, substituted aryl, alkyloxy, substituted alkyloxy, aryloxy, substituted aryloxy, a halogen, or NO₂; R₂ can be OH, or an ester group; R₃ can be a pyridine, a substituted pyridine, an N-oxypyridine, an aryl group, or a substituted aryl group, and wherein in formula II: R₄ can be a phenyl, a 2,4-dinitrophenyl, a benzoyl, a 2-hydroxybenzoyl, a 4-nitrobenzoyl, a pyridine-2-carbonyl, a pyridine-3-carbonyl, a pyridine-4-carbonyl, a 2-hydroxynicotinoyl, a 1-oxidopyridine-3-carbonyl, or —(O═S═O)—R₅; and R₅ can be a 2-naphthyl, a 4-methylphenyl, a 4-methoxyphenyl, a 4-bromophenyl, or a 4-nitrophenyl, and wherein in formula III: R₃ can be a pyridine, a substituted pyridine, an N-oxypyridine, an aryl group, or a substituted aryl group, wherein said composition can be formulated to deliver an effective amount of the composition to a patient in need of relief from an infection or colonization by a microorganism, wherein the microorganism can be a fungal strain or a parasitic protozoa, and wherein the composition further comprises a pharmaceutically acceptable carrier.

In some embodiments of this aspect of the disclosure, the composition can be formulated for the pharmaceutically acceptable administration of the compound to a patient in need thereof by a route selected from the group consisting of an oral route, an intravenous route, a subcutaneous route, a peritoneal route, a topical route, and a vaginal route.

In some embodiments of this aspect of the disclosure, said composition can be formulated to reduce the proliferation of a fungal species.

In some embodiments of this aspect of the disclosure, the fungal strain can be a Candida species.

In some embodiments of this aspect of the disclosure, said composition can be formulated to reduce the proliferation of a protozoal parasite species.

In some embodiments of this aspect of the disclosure, said protozoal parasite species can be Trypanosoma species or a Leishmania species.

Yet another aspect of the disclosure encompasses embodiments of a method of reducing the proliferation of a fungal or protozoal parasite strain in an animal or human patient comprising delivering to an animal or human patient in need thereof a compound or a pharmaceutically acceptable composition comprising said compound according to the present disclosure.

In some embodiments of this aspect of the disclosure said pharmaceutically acceptable composition can be formulated to reduce the proliferation of a fungal species.

In some embodiments of this aspect of the disclosure the fungal strain can be a Candida species.

In some embodiments of this aspect of the disclosure said pharmaceutically acceptable composition can be formulated to reduce the proliferation of a protozoal parasite species.

In some embodiments of this aspect of the disclosure said protozoal parasite species can be Trypanosoma species or a Leishmania species.

It should be noted that ratios, concentrations, amounts, and other numerical data may be expressed herein in a range format.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

Chemistry Experimental: Thin-layer chromatographic analysis (TLC) was performed using silica gel on aluminum foil glass plates and was detected under ultraviolet (UV) light. The ¹H and ¹³C NMR spectra were run on Varian 400 MHz Unity instruments in DMSO-d₆. The solvent signals were used as internal NMR chemical shift references. All products were purified by crystallization from ethanol. If necessary, intermediate esters in preparation of hydrazides (HD1-HD8, Scheme 1, FIG. 1) were purified by short silica gel (40-70 mm) filtration with various mixtures of ethyl acetate and dichloromethane as eluents. Silica gel was purchased from Sorbent Technologies. Substituted phenylhydrazines were prepared from corresponding anilines by following an existing preparation procedure (Vogel Al. A textbook of practical organic chemistry including qualitative organic analysis. 3rd ed. Wiley; 1966.636-637 p). All solvents were purchased from Fisher Scientific, all reagents were purchased from Sigma-Aldrich and were analytical grade.

### Example 2

Preparation 5-nitrosalycilaldehyde: Acetic acid (20 ml) was placed in a large (190×100 mm) crystallization dish equipped with a magnetic stirrer and placed under a fume hood with aqueous sodium carbonate and a nitrogen oxide trap. This acetic acid salicylaldehyde (12.2 g; 0.1 mol) was added to concentrated nitric acid (80 ml). The resulting dark red reaction mixture was stirred at room temperature for approximately 10 minutes, when the reaction started to spontaneously heat up forming a large quantity of nitric oxide (If four times larger reaction scale is used under these conditions, the reaction mixture will explode due to the reaction's exothermicity). The mixture was stirred at room temperature for additional hour; no cooling of the reaction mixture was necessary. During this period, the color of the reaction mixture changed from dark red to orange. This reaction mixture was then poured on crushed ice (400 g) and left at room temperature for one hour. The resulting yellow suspension was filtered and the solid precipitate was collected. The precipitate was washed with ice cold water (3×20 ml) and −5° C. cold methanol (3×15 ml), then dried at room temperature to give pure product (14.6 g; 82%).

### Example 3

Preparation of 2-Hydroxybenzohydrazide (HD2): An ethanol (300 ml) suspension of salicylic acid (13.8 g; 0.1 mol) and strongly acidic ion-exchange resin Amberlyst-15 (5 g) was stirred with refluxing for three days. Insoluble catalyst was separated by filtration, and washed with ethanol (3×20 ml). Combined ethanol filtrates were mixed with hydrazine hydrate (20 ml; 20.5 g; 0.4 mol) and refluxed with slow solvent distillation by using a modified Hickman still apparatus (Scheme 3, FIG. 3). After three hours of refluxing, the volume of the reaction mixture was reduced to approximately 50 ml and white precipitate started to form. The white suspension was cooled to room temperature and then left at −5° C. for one hour. Insoluble product was separated by filtration, washed with ice cold ethanol, and dried on air to give pure product (13.2 g; 87%). ¹H-NMR (DMSO-d₆)) δ 12.3 (1H, broad s, OH), 10.0 (1H, broad s, NH), 7.0 (1H, d of d, J₁=8.4 Hz, J₂=1.6 Hz, 6-H), 7.33 (1H, t, J=8. 4 Hz. 4-H), 6.89 (1H, d, J=8.4 Hz, 3-H), 6.86 (1H, t, J=8.4 Hz, 5-H), and 4.70 (2H, brad s, NH₂) ppm. ¹³C-NMR (DMSO-d₆) δ 168.7, 160.3, 134.1, 127.8, 119.3, 118.0, and 115.1 ppm.

### Example 4

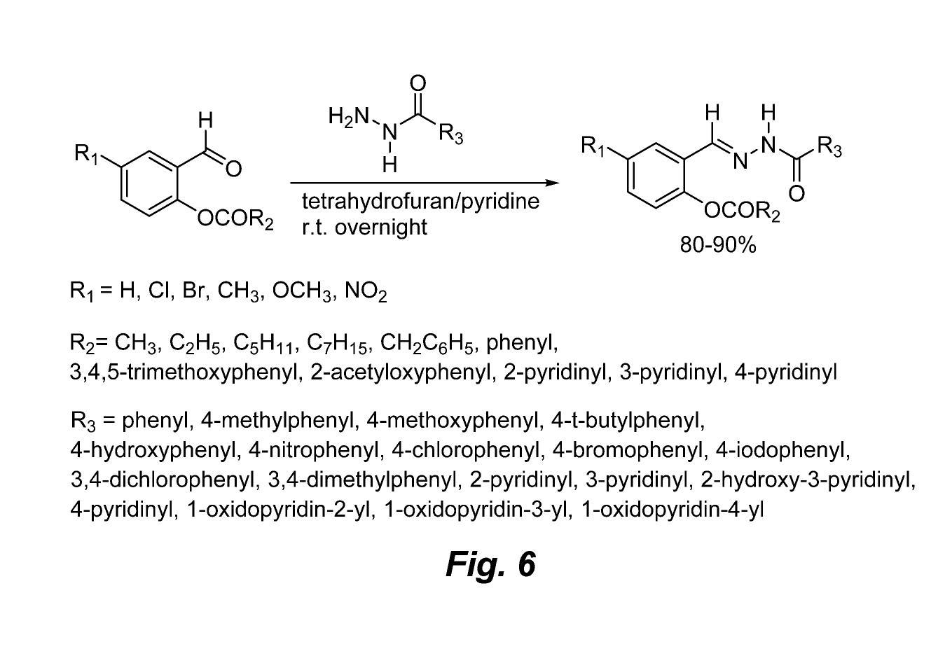

The preparation of prodrugs 1-4, shown in FIG. 5 and FIG. 6, was accomplished in two different synthetic methods. Method A first starts with protection of OH group of 5-substituted salicylaldehydes. The reaction is carried out in pyridine as both a base and reaction solvent with acetic anhydride for acetyl protection, benzoyl chloride for benzoyl protection, and o-acetylbenzoyl chloride for o-acetylbenzoyl protection and in general acid chloride for an ester protection (FIG. 5). Pyridine (30 ml) reaction mixture of substituted salicylaldehyde (0.03 mol) and acetic anhydride (0.036 mol) or corresponding acid chloride (0.036 mol) was stirred at room temperature overnight (approximately 20 h). Volume of the reaction mixture was reduced to a quarter of the original volume under reduced pressure and temperate not exceeding 30° C. Residue was mixed with ice cold water (approximately 100 ml). Resulting solid material was filtered and extensively washed with water (10×30 ml) and dried at 60° C. in vacuum to give pure product in 85-95% yield.

Thus prepared protected salicylaldehydes (0.01 mol) were dissolved in dry tetrahydrofuran (10 ml) and mixed with pyridine (100 ml) solution of corresponding hydrazide. This reaction mixture was left to stir at room temperature overnight. Volume of the reaction mixture was reduced to 10 ml and mixed with ice cold water (100 ml). Insoluble product was separated by filtration, washed with water (5×10 ml) and dried The formed solid material was separated by filtration, washed with ice cold isopropanol and dried at 110° C. for 3 hrs to give pure product in 80-90% yield (as shown in FIG. 5 and FIG. 6). 4-chloro-2-formylphenyl acetate. ¹ H-NMR (CDCl₃) δ 10.07 (1H, s), 7.86 (1H, d, J=2.2 Hz), 7.59 (1H, d of d, J₁=8.6 Hz, J₂=2.2 Hz and 7.17 (1H, d, J=8.6 Hz) ppm.

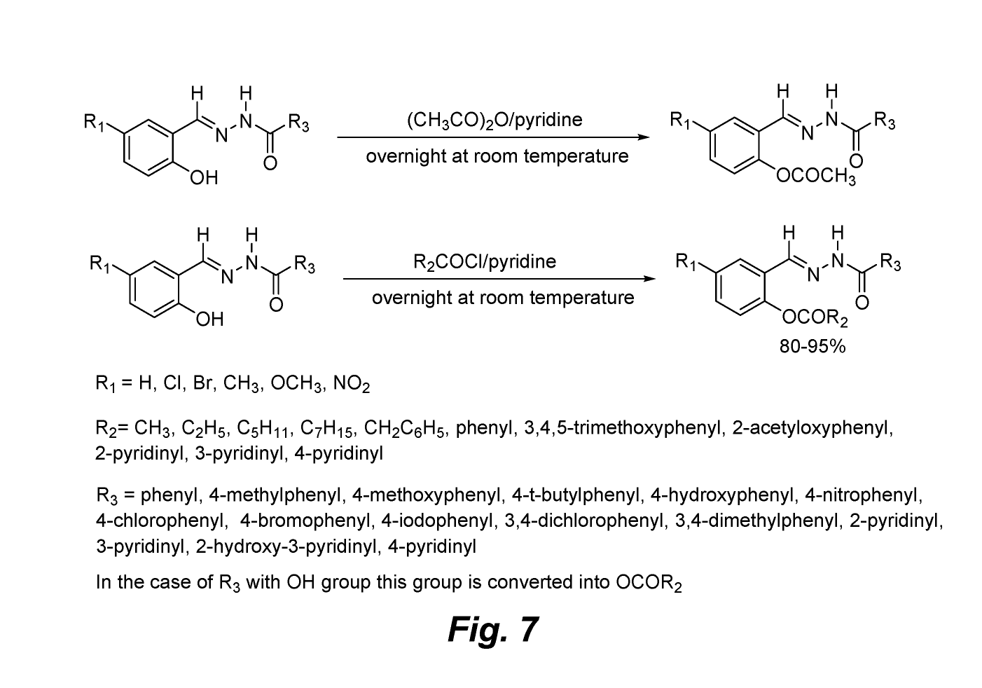

Method B, shown in FIG. 5, involved direct hydroxyl protection of the previously prepared salicyladehyde pyridine hydrazidones in pyridine with acetic anhydride, benzoyl chloride, and o-acetylbenzoyl chloride and in general with corresponding acid chloride. The general procedure developed for preparation acetyl hydrazidones was also applied for preparation benzoyl and o-acetylbenzoyl hydrazidone. Unprotected hydrazidone was dissolved in pyridine (20 mmol in 15 ml pyridine) and acetic anhydride (18 mmol) was added. The resulting reaction mixture was stirred at room temperature overnight. The volume of the reaction mixture was reduced by evaporation at reduced pressure and room temperature to ⅕ of original volume and mixed with ice cold water (200 ml) with stirring. The formed solid product was separated by filtration and extensively washed with ice cold water (5×20 ml). After washing this solid material was dried at 110° C. for 3 hours to give pure product in 80-95% yield (FIG. 7).

(E)-4-chloro-2-((2-picolinoylhydrazono)methyl)phenyl acetate. ¹ H (CDCl₃) δ 11.16 (1H, s), 8.62 (1H, d, J=1.3 Hz), 8.36 (2H, m), 8.05 (1H, d, J=3.2 Hz), 7.94 (1H, t, J=7.5 Hz), 7.53 (1H, m), 7.38 (1H, d of d, J₁=7.6 Hz, J₂=2.2 Hz), 7.11 (1H, d, j=7.6 Hz) and 2.49 (3H, s) ppm.

### Example 5

Picolinohydrazide (HD4): Isolated yield 81% (11.1 g). ¹H-NMR (DMSO-d₆) δ 9.23 (1H, broad s, NH), 8.56 (1H, d, J=4.8 Hz, 6-H), 7.98 (1H, d, J=Hz, 3-C), 7.93 (1H, t=7.6 Hz, 5-H), 7.51 (1H, t, J=4.8 Hz, 4-H), and 4.64 (2H, broad s, NH₂) ppm. ¹³C-NMR (DMSO-d₆) δ 163.4, 150.5, 149.2, 138.3, 126.9, and 122.5 ppm.

### Example 6

Nicotinohydrazide (HD5): Isolated yield 92% (12.6 g). ¹H-NMR (DMSO-d₆) δ 9.99 (1H, s, NH), 8.95 (1H, s, 2-H), 8.64 (1H, m, 4-H), 8.13 (1H, m, 6-H), 7.44 (1H, m, H-5), and 4.61 (2H, NH₂) ppm. ¹³C-NMR (DMSO-d₆) δ 165.2, 152.4, 148.8, 135.4, 129.5, and 124.2 ppm.

### Example 7

Isonicotinohydrazide (HD6). Isolated yield 94% (12.9 g). ¹H-NMR (DMSO-d₆) δ 10.13, 8.66 (2H, d, J=6.0 Hz, 3-H), 7.71 (2H, d, J=6.0 Hz, 2-H), and 4.69 (2H, NH₂) ppm. ¹³C-NMR (DMSO-d₆) δ 164.8, 150.8, 140.9, and 121.7 ppm.

### Example 8

2-Hydroxynicotinohydrazide (HD7). Isolated yield 84% (12.9 g). ¹H-NMR (DMSO-d₆) δ 7.93 (1H, d, J=6.0 Hz), 7.85-7.80 (2H, m), and 8.32 (1H, d, J=9.6 Hz) ppm. ¹³C-NMR (DMSO-d₆) δ 164.3, 163.1, 142.0, 139.4, 137.7, 119.8, and 111.9 ppm.

### Example 9

3-(Hydrazinecarbonyl)pyridine 1-oxide (HD8). Isolated yield (12.1 g; 79%). ¹H-NMR (DMSO-d₆) δ 10.06 (1H, broad s, NH), 8.50 (1H, s, 2-H), 8.32 (1H, d, J=6.4 Hz, 4-H), 7.69 (1H, d, J=8.0 Hz, 6-H), 7.49 (1H, t, J=7.2 Hz, 5-H), and 4.60 (2H, broad s, NH₂) ppm. ¹³C-NMR (DMSO-d₆) δ 162.6, 141.3, 137.9, 133.2, 127.2, and 124.4 ppm.

### Example 10

Preparation of 4-methoxybenzenesulfonohydrazide (SH3). A water (300 ml) solution of hydrazine hydrate (12.5 g; 0.25 mol) was cooled in an ice-water bath to approximately 5° C. A tetrahydrofuran (50 ml) solution containing 4-methoxybenzenesulfonyl chloride (5.16 g, 0.025 mol) was slowly added while stirring slowly. Following this addition, the reaction mixture was stirred at approximately 5° C. for an additional thirty minutes, then the tetrahydrofuran was evaporated at reduced pressure and room temperature. The white solid product was separated by filtration, washed with cold water (3×15 ml) and dried on air overnight to give pure product (4.4 g; 88%). ¹H-NMR (DMSO-d₆) δ 8.20 (1H, s, NH), 7.72 (2H, d, J=8.8 Hz, 2-H), 7.09 (2H, d, J=8.8 Hz), 3.99 (2H, s, NH₂), and 3.81 (3H, s OCH3) ppm. ¹³C-NMR (DMSO-d₆) δ 163.1, 130.5, 130.0, 114.9, and 56.3 ppm.

### Example 11

4-bromobenzenesulfonohydrazide (SH4). Isolated yield 5.6 g (89%). ¹H-NMR (DMSO-d₆) δ 8.47 (1H, s, NH), 7.78 (2H, d, J=8.0 Hz, 2-H), 7.70 (2H, d, J=8.0 Hz, 3-H), and 4.16 (2H, s, NH2) ppm. ¹³C-NMR (DMSO-d₆) δ 138.1, 132.8, 130.4, and 127.3 ppm.

### Example 12

4-Nitrobenzenesulfonohydrazide (SH5). Isolated yield 4.86 g (89%). ¹H-NMR (DMSO-d₆) δ 8.67 (1H, broad s, NH), 8.39 (2H, d, J=7.6 Hz, 3-H), 8.04 (2H, d, J=7.6 Hz, 2-H), and 4.30 (2H, broad s, NH₂) ppm. ¹³C-NMR (DMSO-d₆) δ 150.4, 144.8, 129.9, and 124.9 ppm.

### Example 13

2-Naphthalenesulfonohydrazide (SH6). Isolated yield 5.2 g (93%). ¹H-NMR (DMSO-d₆) δ 8.53 (1H, s, NH), 8.47 (1H, s), 8.12 (2H, m), 8.01 (1H, d, J=7.6 Hz), 7.84 (1H, m), 7.67 (2H, m), and 4.00 (2H, s, NH₂) ppm. ¹³C-NMR (DMSO-d₆) δ 135.9, 135.1, 132.4, 129.9, 129.8, 129.7, 129.4, 128.5, 128.2, and 123.8 pmm.

### Example 14

Preparation of (E)-N′-(2-hydroxybenzylidene)picolinohydrazide (AR1). An ethanol (15 ml) mixture of salycilaldehyde (122 mg; 1 mmol) and picolinohydrazide (137 mg; 1 mmol) was refluxed with slow solvent distillation in modified Hickman still (Scheme 3, FIG. 3A). After the volume of the reaction mixture was reduced to about 3-5 ml white precipitate started to form. This suspension was left at room temperature for one hour followed by an additional hour at 0° C. Insoluble product was removed by filtration, washed with ice cold ethanol (3×2 ml) and dried on air to give pure product in 91% (220 mg) yield. ¹H-NMR (DMSO-d₆) δ 12.51 (1H, s, NH), 11.42 (1H, s, OH), 8.83 (1H, s, CH═N), 8.71 (1H, d, J=4.4 Hz, picolino 3-H), 8.12 (1H, d, J=8.0 Hz, picolino 6-H), 8.04 (1H, t, J=8.0 Hz, picolino 5-H), 7.66 (1H, m, picolino 4-H), 7.45 (1H, d, J=7.6 Hz, salicylic 6-H), 7.29 (1H, t, J=8.0 Hz, salicylic 4-H) and 6.92 (2H, m, salicylic 3-H and 5-H) ppm. ¹³C-NMR (DMSO-d₆) δ 161.1, 158.4, 150.8, 149.8, 149.3, 138.8, 132.2, 130.6, 127.9, 123.5, 120.1, 119.3, and 117.2 ppm.

### Example 15

(E)-N′-(2-Hydroxy-5-methylbenzylidene)picolinohydrazide (AR2). Isolated yield 240 mg (94%). ¹H-NMR (DMSO-d₆) δ 12.47 (1H, s, NH), 11.14 (1H, s, OH), 8.77 (1H, s, CH═N), 8.71 (1H, d, J=4.8 Hz, picolino 3-H), 8.12 (1H, d, J=7.6 Hz, picolino 6-H), 8.05 (1H, t, J=8.0 Hz, picolino 5-H), 7.67 (1H, t, J=6.0 Hz, picolino 4-H), 7.26 (1H, s, salicylic 6-H), 7.10 (1H, d, J=8.4 Hz, salicylic 4-H), 6.82 (1H, d, J=8.4 Hz, salicylic 3-H), and 2.24 (3H, s, CH₃) ppm. ¹³C-NMR (DMSO-d₆) δ 156.2, 150.7, 149.9, 149.3, 138.8, 132.9, 130.5, 128.6, 127.9, 123.5, 118.9, 117.0, 115.6, and 20.6 ppm.

### Example 16

(E)-N′-(2-Hydroxy-5-methoxybenzylidene)picolinohydrazide (AR3). Isolated yield 250 mg (92%). ¹H-NMR (DMSO-d₆) δ 12.47 (1H, s, NH), 10.80 (1H, s, OH), 8.79 (1H, s, CH═N), 8.71 (1H, d, J=4.8 Hz, picolino 3-H), 8.11 (1H, d, J=7.6 Hz, picolino 6-H), 8.07 (1H, t, J=7.6 Hz, picolino 5-H), 7.66 (1H, m, picolino 4-H), 7.26 (1H, s, salicylic 6-H), 7.03 (1H, d, J=2.8 Hz, salicylic 6-H), 6.91 (1H, d of d, J₁=8.8 Hz, J₂=3.2 Hz, salicylic 4-H), 6.85 (1H, d, J=8.8 Hz), and 3.72 (3H, s, CH₃) ppm. ¹³C-NMR (DMSO-d₆) δ 161.2, 152.8, 152.4, 150.0, 149.3, 138.8, 135.5, 127.8, 123.5, 119.5, 119.1, 118.0, 113.3, and 56.2 ppm.

### Example 17

(E)-N′-(5-Chloro-2-hydroxybenzylidene)picolinohydrazide (AR4). Isolated yield 255 mg (92%). ¹H-NMR (DMSO-d₆) δ 12.56 (1H, s, NH), 11.33 (1H, s, OH), 8.80 (1H, s, CH═N), 8.70 (1H, d, J=4.8 Hz, picolino 3-H), 8.12 (1H, d, J=7.6 Hz, picolino 6-H), 8.04 (1H, t of d, J₁=7.6 Hz, J₂=1.6 Hz, picolino 5-H), 7.65 (1H, m, picolino 4-H), 7.57 (1H, d, J=2.8 Hz, salicylic 6-H, 7.30 (1H, d of d, J₁=8.8 Hz, J₂=2.8 Hz, salicylic 4-H), and 6.93 (1H, d, J=8.8 Hz) ppm. ¹³C-NMR (DMSO-d₆) δ 161.3, 156.9, 149.8, 149.3, 148.3, 138.7, 131.6, 128.6, 127.9, 123.6, 123.5, 121.3, and 118.9 ppm.

### Example 18

(E)-N′-(5-bromo-2-hydroxybenzylidene)picolinohydrazide (AR5). Isolated yield 300 mg (94%). ¹H-NMR (DMSO-d₆) δ 12.55 (1H, s, NH), 11.34 (1H, s, OH), 8.79 (1H, s, CH═N), 8.69 (1H, d, J=4 Hz, picolino 3-H), 8.11 (1H, d, J=7.6 Hz, picolino 6-H), 8.03 (1H, t, J=7.6 Hz, picolino 5-H), 7.69 (1H, d, J=2.4 Hz, salicylic 6-H), 7.65 (1H, m, picolino 4-H), 7.40 (1H, d od d, J₁=8.8 Hz, J₂=2.4 Hz, salicylic 4-H), and 6.88 (1H, d, J=8.8 Hz, salicylic 3-H) ppm. ¹³C-NMR (DMSO-d₆) δ 161.3, 157.3, 149.8, 149.3, 148.1, 138.7, 134.4, 131.5, 127.9, 123.6, 121.9, 119.4, and 111.1 ppm.

### Example 19

(E)-N′-(2-hydroxy-5-nitrobenzylidene)picolinohydrazide (AR6). Isolated yield 260 mg (90%). ¹H-NMR (DMSO-d₆) δ 12.62 (1H, s, NH), 12.30 (1H, s, OH), 8.88 (1H, s, CH═N), 8.68 (1H, d, J=4 Hz, picolino 3-H), 8.45 (1H, d, J=2.8 Hz, salicylic 6-H), 8.11 (2H, m), 8.03 (1H, m), 7.64 (1H, m) and 7.40 (1H, d, J=9.2 Hz, salicylic 3-H) ppm. ¹³C-NMR (DMSO-d₆) δ 163.5, 161.4, 149.7, 149.3, 146.8, 140.5, 138.7, 127.3, 127.3, 124.8, 123.6, 120.5, and 117.8 ppm.

### Example 20

TABLE 1
Antifungal Activity of N′-(2-hydroxybenzylidene)picolinohydrazides

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | Com- | | TW1 | TW17 | ATCC No. | ATCC No. |
| R | pound | Yield | (μg/ml) | (μg/ml) | 10231 | 48435 |
| H | AR1 | 91% | 62 | NA | 16 | 8 |
| CH₃ | AR2 | 94% | 4 | 8 | 2 | 2 |
| CH₃O | AR3 | 92% | 8 | 31 | 4 | 1 |
| Cl | AR4 | 92% | 4 | 8 | 1 | 1 |
| Br | AR5 | 94% | 2 | 4 | 1 | 4 |
| NO₂ | AR6 | 90% | 8 | 8 | 4 | 8 |

¹Azole-susceptible;
²Azole-resistant;
³Azole-susceptible;
⁴Azole-resistant

These derivatives are all hydrazones of salicylaldehyde and five-substituted salicylaldehyde. All of the synthesized derivatives in this group consistently inhibited fungal growth of the azole-susceptible fungal species at very low concentrations (approximately 3-15 μM-Table 1). In addition, 4 of the 6 derivatives (namely AR2, AR4, AR5, and AR6-Table 1) inhibited fungal growth in the azole-resistant fungi TW17 and C. glabrata at a concentration range of 3-30 μM.

### Example 21

The position of the nitrogen in the pyridine ring had little effect on the ability of the resulting hydrazone analog to act as a fungal growth inhibitor. 5-substituted salicylaldehyde hydrazones of nicotinohydrazide (Table 2) also consistently inhibited fungal growth, even though several derivatives were less active against azole-resistant isolates (AR8 and AR12).

TABLE 2
Antifungal Activity of N′-(2-hydroxybenzylidene)nicotinohydrazides

| | | | | | ³MIC₈₀ C. albicans | ⁴MIC₈₀ C. glabrata |
|---|---|---|---|---|---|---|
| | | | ¹MIC₈₀ | ²MIC₈₀ | (μg/ml) | (μg/ml) |
| | | | TW1 | TW17 | ATCC no. | ATCC no. |
| R | Compound | Yield | (μg/ml) | (μg/ml) | 10231 | 48435 |
| H | AR7 | 91% | 4 | 31 | 4 | 16 |
| CH₃ | AR8 | 93% | 16 | NA | 4 | 8 |
| CH₃O | AR9 | 86% | 16 | 4 | 62 | 4 |
| Cl | AR10 | 91% | 8 | 62 | 4 | 8 |
| Br | AR11 | 92% | 8 | 16 | 4 | 16 |
| NO₂ | AR12 | 86% | 8 | 8 | 4 | NA |

¹Azole-susceptible;
²Azole-resistant;
³Azole-susceptible;
⁴Azole-resistant

### Example 22

Substituted salicylaldehyde hydrazones of isonicotinohydrazide all possessed antifungal activity with the most potent again being the 5-nitrosalicylaldehyde hydrazone (Table 3; AR18).

TABLE 3
Antifungal Activity of N′-(2-hydroxybenzylidene)isonicotinohydrazides

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | Com- | | TW1 | TW17 | ATCC no. | ATCC no. |
| R | pound | Yield | (μg/ml) | (μg/ml) | 10231 | 48435 |
| H | AR13 | 90% | 4 | 31 | 8 | 16 |
| CH₃ | AR14 | 92% | 16 | 31 | 4 | 31 |
| CH₃O | AR15 | 91% | 16 | 16 | 4 | 4 |
| Cl | AR16 | 94% | 16 | 125 | 31 | 16 |
| Br | AR17 | 97% | 16 | 125 | 16 | 64 |
| NO₂ | AR18 | 86% | 8 | 8 | 4 | 31 |

¹Azole-susceptible;
²Azole-resistant;
³Azole-susceptible;
⁴Azole-resistant

### Example 23

To determine the effects of an OH-group or oxidizing pyridine moiety on fungal growth inhibition, these two functionalities were individually incorporated on the nicotinohydrazide derivatives. Unlike the previous nicotinohydrazide tested where all derivatives were active (see Table 2) the incorporation of the OH-functionality in the two position of the pyridine ring led to inconsistent results. Introducing the hydroxyl group diminished or even eliminated the antifungal activity previously observed.

TABLE 4
Antifungal Activity of
2-hydroxy-N′-(2-hydroxybenzylidene)nicotinohydrazides

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | Com- | | TW1 | TW17 | ATCC no. | ATCC no. |
| R | pound | Yield | (μg/ml) | (μg/ml) | 10231 | 48435 |
| H | AR19 | 89% | 4 | 4 | NA | 1 |
| CH₃ | AR20 | 87% | NA | NA | NA | 2 |
| Cl | AR21 | 91% | NA | NA | NA | NA |
| Br | AR22 | 93% | NA | NA | NA | 0.5 |

¹Azole-susceptible;
²Azole-resistant;
³Azole-susceptible;
⁴Azole-resistant

However, growth inhibition was observed at very low concentrations across all Candida albicans isolates treated with AR19. In addition, AR22 was one of the most potent compounds against Candida glabrata.

### Example 24

In the case of the pyridine oxides presented in Table 5, activity of the analogs was consistent, with all derivatives exhibiting inhibition of fungal growth, regardless of the azole-sensitivity or resistance, but the general MICs were higher (Table 5).

TABLE 5
Antifungal Activity of
3-(2-(2-hydroxybenzylidene)hydrazinecarbonyl)pyridine 1-oxides

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | Com- | | TW1 | TW17 | ATCC no. | ATCC no. |
| R | pound | Yield | (μg/ml) | (μg/ml) | 10231 | 48435 |
| H | AR23 | 87% | 31 | 31 | 31 | 16 |
| CH₃ | AR24 | 89% | 16 | 16 | 16 | 31 |
| CH₃O | AR25 | 88% | 62 | 31 | 16 | 4 |
| Cl | AR26 | 91% | 31 | 31 | 16 | 4 |
| Br | AR27 | 93% | 8 | 8 | 16 | 4 |
| NO₂ | AR28 | 85% | 4 | 2 | 8 | 4 |

¹Azole-susceptible;
²Azole-resistant;
³Azole-susceptible;
⁴Azole-resistant

The most potent analog in this class against azole-resistant TW17 was AR28 (activity of 2 μM).

### Example 25

While not wishing to be bound by anyone theory, the pyridine structural motif might be essential for antifungal activity, particularly for azole-resistant isolates. Various hydrazones of 2-pyridinecarbaldehyde were prepared for further analysis of this motif (Table 6 and Table 7).

TABLE 6
Antifungal activity of phenyl and aroyl N′-substituted 2-(hydrazonomethyl)pyridines

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | | | TW1 | TW17 | ATCC no. | ATCC no. |
| R | Compound | Yield | (μg/ml) | (μg/ml) | 10231 | 48435 |
| Phenyl | AR29 | 93% | 16 | 16 | 16 | 31 |
| 2,4-dinitrophenyl | AR30 | 91% | NA | NA | NA | NA |
| benzoyl | AR31 | 90% | 31 | 62 | 62 | 62 |
| 2-hydroxybenzoyl | AR32 | 88% | 31 | 62 | 31 | 4 |
| 4-nitrobenzoyl | AR33 | 86% | NA | NA | NA | NA |
| Pyridine-2-carbonyl | AR34 | 92% | 16 | 16 | 31 | 16 |
| Pyridine-3-carbonyl | AR35 | 93% | 62 | 62 | 125 | 125 |
| Pyridine-4-carbonyl | AR36 | 91% | 62 | 125 | 125 | 125 |
| 2-hydroxynicotinoyl | AR37 | 88% | NA | NA | NA | NA |
| 1-oxidopyridine-3- | AR38 | 90% | 62 | 125 | 125 | 125 |
| carbonyl | | | | | | |

¹Azole-susceptible;
²Azole-resistant;
³Azole-susceptible;
⁴Azole-resistant

TABLE 7
Antifungal activity of aryl substituted N′-(2-pyridinylmethylene)sulfonic hydrazides

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | | | TW1 | TW17 | ATCC no. | ATCC no. |
| R | Compound | Yield | (μg/ml) | (μg/ml) | 10231 | 48435 |
| 2-naphthyl | AR39 | 91% | NA | NA | NA | NA |
| 4-methylphenyl | AR40 | 88% | NA | NA | NA | NA |
| 4-methoxyphenyl | AR41 | 93% | NA | NA | NA | NA |
| 4-bromophenyl | AR42 | 97% | NA | NA | NA | NA |
| 4-nitrophenyl | AR43 | 91% | NA | NA | NA | NA |

¹Azole-susceptible;
²Azole-resistant;
³Azole-susceptible;
⁴Azole-resistant

Each of these analogs lacks the corresponding salicylaldehyde moiety present on the active derivatives presented in Tables 1-5. The phenylhydrazone AR29, and the picolinohydrazide AR34 (Table 6) showed moderate activity, likely due to structural similarities to compounds shown in Table 1, but in general, the loss of the salicyl moiety resulted in a significant loss of antifungal activity. Furthermore, sulfohydrazide derivatives showed no antifungal activity (Table 7) which is consistent with published data showing that compounds bearing a sulfonyl group are essentially inactive (Backes et al., Bioorg. Med. Chem. (2014) 22: 4629-4636).

### Example 26

Because phenylhydrazone AR29 showed moderate activity, two groups of phenylhydrazones were prepared and tested to determine if it was possible to increase the antifungal activity. One set of analogs was synthesized from 3-pyridinecarbaldehyde (Table 8) and the other from 4-pyridinecarbaldehyde (Table 9).

TABLE 8
Antifungal activity of aryl substituted 3-(hydrazonomethyl)pyridines

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | | | TW1 | TW17 | ATCC no. | ATCC no. |
| R | Compound | Yield | (μg/ml) | (μg/ml) | 10231 | 48435 |
| Phenyl | AR44 | 93% | 16 | 8 | 16 | 16 |
| 2,4-dinitrophenyl | AR45 | 96% | NA | NA | NA | NA |
| Benzoyl | AR46 | 91% | NA | NA | NA | NA |
| 4-nitrobenzoyl | AR47 | 86% | NA | NA | NA | NA |

¹Azole-susceptible;
²Azole-resistant;
³Azole-susceptible;
⁴Azole-resistant

TABLE 9
Antifungal activity of aryl substituted 4-(hydrazonomethyl)pyridines

| | | | | | ³MIC₈₀ | ⁴MIC₈₀ |
|---|---|---|---|---|---|---|
| | | | | | C. albicans | C. glabrata |
| | | | ¹MIC₈₀ | ²MIC₈₀ | (μg/ml) | (μg/ml) |
| | | | TW1 | TW17 | ATCC no. | ATCC no. |
| R | Compound | Yield | (μg/ml) | (μg/ml) | 10231 | 48435 |
| Phenyl | AR48 | 92% | 16 | 8 | 31 | 4 |
| 2,4-dinitrophenyl | AR49 | 95% | NA | NA | NA | NA |
| Benzoyl | AR50 | 89% | NA | NA | NA | NA |
| 4-nitrobenzoyl | AR51 | 87% | NA | NA | NA | NA |

¹Azole-susceptible;
²Azole-resistant;
³Azole-susceptible;
⁴Azole-resistant

As in the case of 2-pyridinecarboldehyde, moderate antifungal activity was observed with phenylhydrazones AR44 and AR48, but not with any other derivatives. Based on these data, the most consistent results with respect to growth inhibition of both azole-susceptible and azole-resistant fungal isolates were obtained with hydrazones bearing both salicylaldehyde and pyridine structural motifs.

### Example 27

### Synergy Studies-Checkerboard Assay

Considering that two different modes of activity (cidal and static) were observed depending on the structural motifs present in our analogs, we performed a series of checkerboard assays to determine if representative active compounds were in fact acting in synergy with the azole, fluconazole. Each of these assays tested multiple concentrations of the selected analog in the presence of multiple concentrations of fluconazole. All checkerboard assays were prepared in 96-well plates. 200 μl of previously prepared drug solutions were added to each well in column 2. Using a multichannel pipette, 100 μl from each well in column 2 was serially diluted (1:2) horizontally across the plate, stopping at column 10. Contents of each well were mixed thoroughly. Column 11 was reserved for fluconazole MIC control and contained no other drug. 100 μl of media was added to each well, column 2-10. Each well was mixed thoroughly with a multichannel pipette. 100 μl was then removed and discarded from these wells. 100 μl of previously prepared fluconazole dilution was added to each well in row B. Using a multichannel pipette, 100 μl from each well was serially diluted (1:2) vertically down the plate, stopping at row F. Contents of each well were mixed thoroughly. Row G was reserved for tested drug MIC control and contained no fluconazole. Well G11 contained neither drug and served as a drug-free growth control. The final 100 μl from each well in row F was discarded.

The fungal samples were prepared from glycerol stocks of Candida albicans ATCC Accession No. 10231. The inoculum's concentration was adjusted to a McFarland standard between 0.08 and 0.12. Inoculum was then diluted at a ratio of 1:37.5 in buffered media. 100 μl of inoculum-media solution was added to each well. Following inoculation, the 96-well plates were wrapped in parafilm to prevent evaporation and incubated for overnight at 35° C. After 18 h, the wells showing growth were recorded.

Synergistic activity was determined based on the MIC read from these plates. The fractional inhibitory concentration (FIC) for a given well is the ratio of the minimum inhibitory concentration of one drug A in the presence another drug B to the minimum inhibitory concentration with only drug A. The FIC was calculated for the test drug in each of the wells along the growth/no-growth interface at varying concentrations of fluconazole, then these values were averaged to determine the average FIC for the test drug. The same process was used to calculate the average FIC for fluconazole at varying concentrations of the test drug. The sum of these two FICs gives the FIC index which can be used to determine the synergistic effect between the two drugs (Hsieh et al., Diagn. Microbiol. Infect. Dis. (1993) 16: 343-349). An FIC index value less than 1 indicated synergistic interactions between the two drugs while an FIC index value greater than or equal to 1 indicated antagonistic interactions. Table 10 shows the results from our checkerboard assays, indicating no synergy exists between fluconazole and our new analogs.

TABLE 10
Fractional Inhibitory Concentrations (FIC)
for select analogs with fluconazole

| | FIC | FIC | FIC | | Cidal vs |
|---|---|---|---|---|---|
| Compound | Fluc | Test | Index | Activity | Static |
| AR2 | 1.00 | 1.00 | 2.00 | Antagonistic | Static |
| AR5 | 1.00 | 0.90 | 1.90 | Antagonistic | Cidal |
| AR6 | 1.00 | 1.00 | 2.00 | Antagonistic | Static |
| AR12 | 1.00 | 1.00 | 2.00 | Antagonistic | Static |
| AR18 | 1.00 | 1.00 | 2.00 | Antagonistic | Static |
| AR27 | 0.92 | 1.00 | 1.92 | Antagonistic | Static |
| AR28 | 1.00 | 1.00 | 2.00 | Antagonistic | Static |
| AR44 | 1.00 | 0.90 | 1.90 | Antagonistic | Cidal |
| AR48 | 3.87 | 0.80 | 4.67 | Antagonistic | Cidal |

1. A compound having the formula I, II, or III, or a hydrate or pharmaceutically acceptable salt thereof: 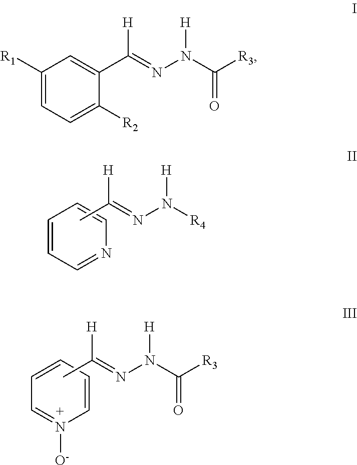
wherein in formula I:
R₁ is H, alkyl, aryl, substituted aryl, alkyloxy, substituted alkyloxy, aryloxy, substituted aryloxy, a halogen, or NO₂;
R₂ is OH, or an ester group;
R₃ is a pyridine, a substituted pyridinyl, an N-oxypyridine, an aryl group, or a substituted aryl group,
with the proviso that when R₂ is OH and R₃ is 2-pyridinyl then R₁ is not H, a methyl, or NO₂, and when R₂ is OH and R₃ is 3-pyridinyl or 4-pyridinyl then R₁ is not H, a methyl, a methoxy, a halogen, or NO₂;
and wherein in formula II:
R₄ is a phenyl, a 2,4-dinitrophenyl, a benzoyl, a 2-hydroxybenzoyl, a 4-nitrobenzoyl, a pyridine-2-carbonyl, a pyridine-3-carbonyl, pyridine-4-carbonyl, a 2-hydroxynicotinoyl, a 1-oxidopyridine-3-carbonyl, or —(O═S═O)—R₅; and
R₅ is a 2-naphthyl, a 4-methylphenyl, a 4-methoxyphenyl, a 4-bromophenyl, or a 4-nitrophenyl,
and wherein in formula III:
R₃ is a pyridine, a substituted pyridine, an N-oxypyridine, a aryl group, or a substituted aryl group.

2. The compound of claim 1, wherein R₂ is an ester having the forty OCOX, wherein X is selected from the group consisting of: methyl, ethyl, cyclopentane cycloheptane, CH₂C₆H₅, phenyl, 3,4,5-trimethoxyphenyl 2-acetyloxphenyl, 2-pyridinyl, 3-pyridiyl, and 4-pyridinyl.

3. The compound of claim 1, wherein R₃ is selected from the group consisting of: phenyl, 4-methylphenyl, 4-methoxyphenyl 4-t-butyiphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3 pyridinyl, 4-pyridinyl, 1-oxidopyridin-2-yl, 1-oxidopyridin-3-yl, and 1-oxidopyridin-4-yl.

4. The conpound of claim 1, wherein the compound has the formula I 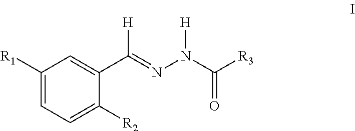
wherein:
R₁ is H, an alkyl, an alkyloxy, a halogen, NO₂;
R₂ is OH, or an ester group;
R₃ is a pyridine, a substituted pyridine, an N-oxypyridine, an aryl group, or a substituted aryl group,
with the proviso that when R₂ is OH and R₃ is 2-pyridinyl then R₁ is not H, a methyl, or NO₂, and when R₂ is OH and R₃ is 3-pyridinyl or 4-pyridinyl then R₁ is not H, a methyl, a methoxy, a halogen, or NO₂.

5. The compound of claim 4. wherein R₂ is an ester eying the formula OCOX, wherein X is selected from the group consisting of: methyl, ethyl, oyclopentane, cycloheptane, CH₂C₆H₅, phenyl, 3,4,5-trimethoxyphenyl, 2-acetyloxyphenyl, 2-pyridinyl, 3-pyridinyl, and 4-pyridinyl.

6. The compound of claim 4, wherein R₃ is selected from the group consisting of: phenyl, 4-methylphenyl, 4-methoxyphenyl, 4-t-butylphenyl, 4-hydroxyphenyl, 4-nitrophenyl, 4-chlorophenyl, 4-bromophenyl, 4-iodophenyl, 3,4-dichlorophenyl, 3,4-dimethylphenyl, 2-pyridinyl, 3-pyridinyl, 2-hydroxy-3-pyridinyl, 4-pyridinyl, 1-oxidopyridin-2-yl, 1-oxidopyridin-3-yl, and 1-oxidopyridin-4-yl.

7. The compound of claim 4, wherein the compound has the formula I, and wherein R₁ is H, CH₃, OCH ₃, Cl, Br, or NO₂ and R₂ is OCOCH₃, OCOC₆H₅, or OCOC₆H₅-o-OCOCH₃.

8. The compound of claim 1, wherein the compound has the formula II: 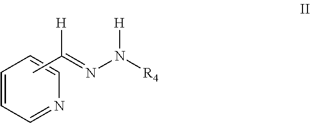
wherein:
R₄ is a phenyl, 4-methylphenyl, 4-methoxyphenyl, 4-t-butylphonyl, 4,-hydroxyphenyl, 4-nitrophenyl, 4-chlorophenyl, 4-bromophenyl, 4-iodophonyl, 3,4-dichlorophenyl, 3,4-dimethylphenyl, 2-pyridinyl, 3-pyridinyl, 2-hydroxy-3-pyridinyl, 4-pyridinyl, 1-ondopyridin-2-yl, 1-oxidopyridin-3-yl, 1-oxidopyridin-4-yl, or —(O═S═O)—R₅; and
R₅ is a 2-naphthyl, a 4-methylphenyl, a 4-methoxyphenyl, a 4-brompphenyl, or a 4-nitrophenyl.

9. The compound of claim 1, wherein the compound has the formula III: 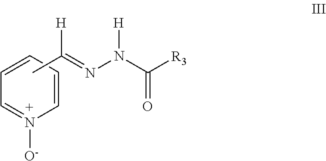
wherein R₃ selected, from the group consisting of: phenyl, 4-methylphenyl, 4-methoxyphenyl, 4-t-butylphonyl, 4-hydroxyphenyl, 4-nitrophenyl, 4-chlorophenyl, 4-bromophenyl, 4-iodophenyl, 3,4,-dichlorophenyl, 3,4-dimethylphenyl, 2-pyridnyl, 3-pyridnyl, 2-hydroxy-3-pyridinyl, 4-pyridinyl, 1-oxidopyridin-2-yl, 1-oxidopyridin-3-yl, and 1-oxidopyridin-4-yl.

10. The compound of claim 1, wherein said compound is selected from the group consisting of

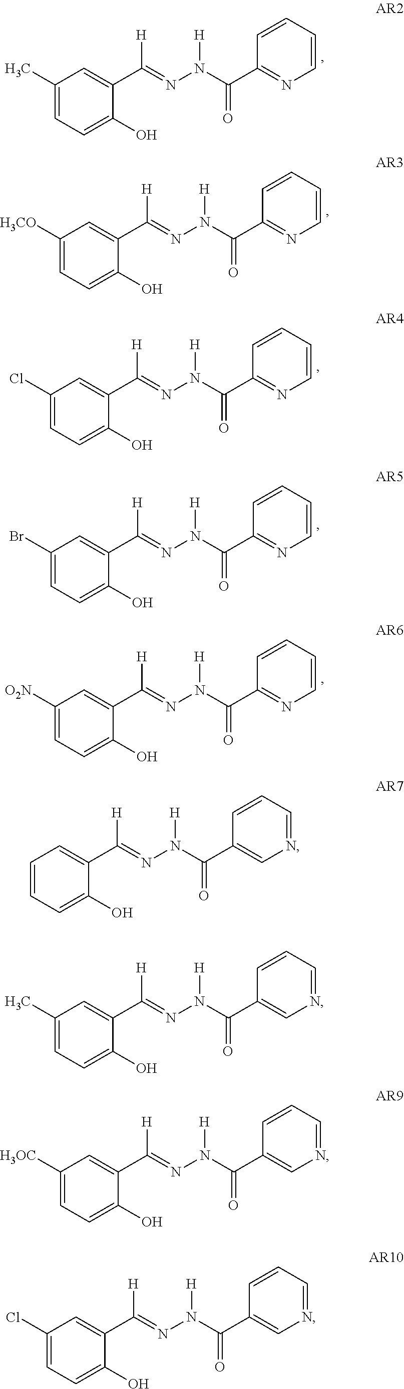

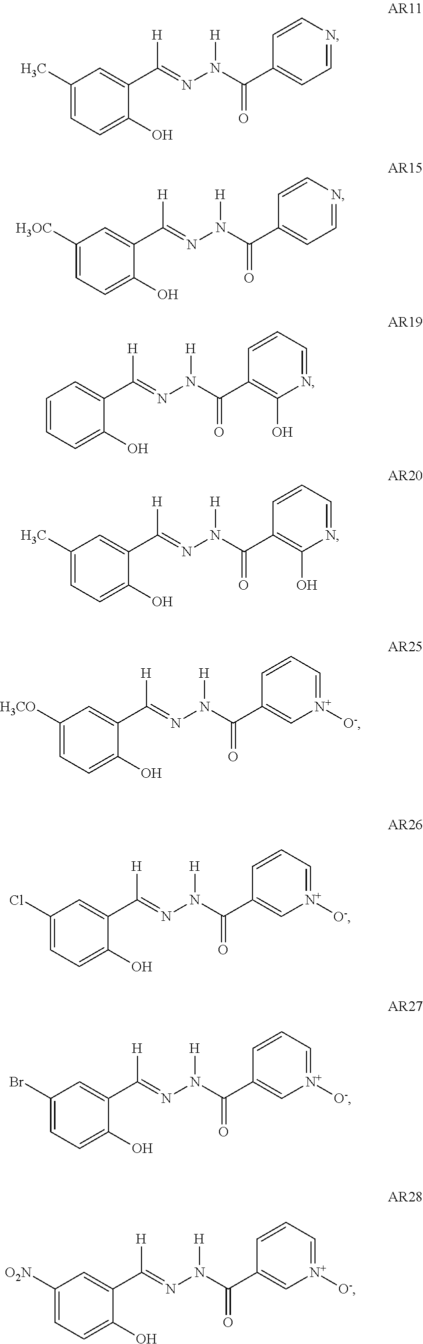

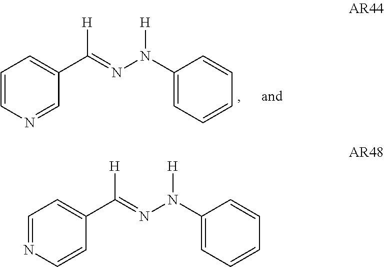 [SMILES: C(=N\Nc1ccccc1)/c1cccnc1 ; C(=N\Nc1ccccc1)/c1ccncc1]

11. A pharmaceutically acceptable composition comprising at least one compound having the formula I, II, or III, or a hydrate pr pharmaceutically acceptable salt thereof: 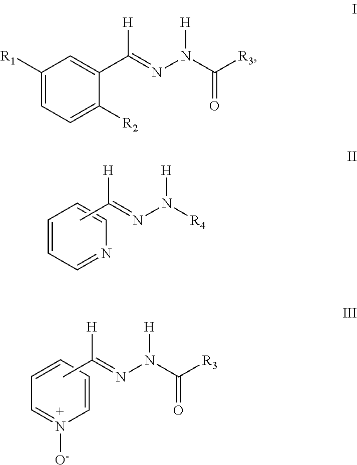
wherein in formula I:
R₁ is H, alkyl, aryl, substituted aryl, alkyloxy, substituted alkyloxy, aryloxy, substituted aryloxy, a halogen, or NO₂;
R₂ is OH, or an ester group;
R₃ is a pyridine, a substituted pyridine, an N-oxypyridine, an aryl group, or a substituted aryl group,
and wherein in formula II:
R₄ is a phenyl, a 2,4-dinitrophenyl, a benzoyl, 2-hydroxybenzoyl, a 4-nitrobenzoyl, a pyridine-2-carbonyl, a pyridine-3-carbonyl, a pyridine-4-carbonyl, a 2-hydroxynicotinoyl, a 1-oxidopyridine-3-carbonyl, or —(O═S═O)—R₅; and
R₅ is a 2-naphthyl, a 4-methylphenyl, a 4-methoxyphenyl, a 4-bromophenyl, or a 4-nitrophenyl,
and wherein in formula III:
R₃ is a pyridine, a substituted pyridine, an N-oxypyridine, an aryl group, or a substituted aryl group, or a hydrate pharmaceutically acceptable salt thereof,
wherein said composition is formulated to deliver an effective amount of the composition to a patient in need of relief from an infection or colonization by a microorganism, wherein the microorganism is a fungal strain or a parasitic of protozoa, and wherein the composition further comprises a pharmaceutically acceptable carrier.

12. The pharmaceutically acceptable antimicrobial composition of claim 11, wherein the composition is formulated for the pharmaceutically acceptable administration of the compound to a patient in need thereof by a route selected from the group consisting of an oral route, an intravenous route, a subcutaneous route, a peritoneal route, a topical route or a vaginal route.

13. The pharmaceutically acceptabie antimicrobial composition of claim 11, wherein said composition is formulated to reduce the proliferation of a fungal species.

14. The pharmaceutically acceptable antimicrobial composition of claim 13, wherein the fungal strain is a Candida species.

15. The pharmaceutically acceptable antimicrobial composition of claim 11, wherein said composition is formulated to reduce the proliferation of a protozoal parasite species.

16. The pharmaceutically a acceptable antimicrobial composition of claim 11, wherein said protozoal parasite species is Trypanosoma species or a Leishmania species.

17. A method of reducing the proliferation of a fungal or protozoal parasite strain in an animal or human patient comprising delivering to an animal or human patient in need thereof a compound or a pharmaceutically acceptable composition comprising said compound according to claim 1.

18. The method of claim 17, wherein said pharmaceutically acceptable composition is formulated to reduce the proliferation of a fungal species.

19. The method of claim 18, wherein the fungal strain is Candida species.

20. The method of claim 17, wherein said pharmaceutically acceptable composition is formulated to reduce the proliferation of a protozoal parasite species.

21. The method of claim 20, wherein said protozoal parasite species is Trypanosoma species or a Leishmania species.
